(19) United States
(12) Patent Application Publication
(10) Pub. No.: US 2026/0207624 A1
(43) Pub. Date: Jul. 23, 2026

(54) COADMINISTRATION OF ESTROGEN WITH ESTROGEN RECEPTOR MODULATOR OR DEGRADER OR ER TARGETED PROTAC FOR THE TREATMENT OF ESTROGEN RESPONSIVE CANCERS

(71) Applicant: Duke University, Durham, NC (US)

(72) Inventors: Donald P. McDonnell, Chapel Hill, NC (US); Suzanne E. Wardell, Durham, NC (US)

(21) Appl. No.: 19/140,980

(22) PCT Filed: Dec. 21, 2023
(86) PCT No.: PCT/US2023/085575

§ 371 Date: Jun. 18, 2025

Related U.S. Application Data
(60) Provisional application No. 63/476,606, filed on Dec. 21, 2022.
(60) Provisional application No. 63/581,259, filed on Sep. 7, 2023.

(51) Int. Cl. A61K 31/565; A61K 31/085; A61K 31/138; A61K 31/40; A61K 31/4439; A61K 31/4535; A61K 31/55; A61K 31/566; A61K 31/568; A61K 45/06; A61P 35/00

(52) U.S. Cl. CPC A61K 31/565; A61K 31/085; A61K 31/138; A61K 31/40; A61K 31/4439; A61K 31/4535; A61K 31/55; A61K 31/566; A61K 31/568; A61K 45/06; A61P 35/00

(57) ABSTRACT

Disclosed herein are compositions and methods for treating cancer. The methods may include administering to a subject at least one brain-excluded estrogen receptor (ER) modulating drug. The methods may further include administering to the subject at least one brain-penetrating ER agonist or at least one brain-penetrating androgen receptor (AR) modulator. The brain-excluded ER modulating drug may be administered to a subject at a dose that results in from zero to minimal brain exposure.

### CROSS-REFERENCE TO RELATED APPLICATIONS

This application claims priority to U.S. Provisional Patent Application No. 63/476,606, filed Dec. 21, 2022, and U.S. Provisional Patent Application No. 63/581,259, filed Sep. 7, 2023, the entire contents of each of which are hereby incorporated by reference.

### STATEMENT REGARDING FEDERALLY SPONSORED RESEARCH

This invention was made with government support under grant BC170954 and grant W81XWH-18-1-0064 awarded by the United States Department of Defense. The government has certain rights in the invention.

### FIELD

This disclosure relates to compositions and methods for treating cancer, including estrogen receptor (ER) modulating drugs.

### INTRODUCTION

Estrogens are widely recognized as promoting the growth and metastasis of breast cancer tumors. Estrogens mediate their physiological actions in cells through the classical nuclear estrogen receptors (ERs; ERα and ERβ) and through the non-classical G-protein coupled receptor GPER1 (also referred to as GPR30). ERs have also been shown to be expressed in several different cell types within the tumor microenvironment, and other cancer types can show a tumor growth response to estrogens. Modulation of host cells within the tumor microenvironment is one mechanism by which estrogens can impact tumor growth and metastasis, but the extent to which ERs play a role in tumor progression remains to be determined. The differential expression and activity of different ERs in different cells within tumors and in cells that act upon tumor cells may determine tumor response to ER modulators. Indeed, 17β-estradiol (E2) working through ERα expressed in endothelial cells in the tumor microenvironment has been shown to induce tumor growth by improving tumor angiogenesis and protecting tumor cells against hypoxia and necrosis. Further, ER actions have been studied in different immune cell types in different diseases, but the extent to which ER influences immune cell biology within the tumor microenvironment has not been examined in detail. Recently, it has been demonstrated in ovarian cancer that E2 can create an immune suppressive tumor microenvironment (TME) by promoting the mobilization of myeloid-derived suppressor cells (MDSC) from bone, which function to suppress tumor immunity and increase tumor growth. While this study demonstrates that ER function is important for MDSC mobilization, the tumor microenvironment is infiltrated with multiple other myeloid cell types such as dendritic cells (DCs), monocytes, and tumor associated macrophages, all of which impact tumor immunity. ERs have been shown to play a critical role in development and functionality of these myeloid cell types, however, the extent to which ER function regulates myeloid cell-T cell crosstalk within the TME is not known. There is a need for improved therapies for estrogen responsive cancers.

While considerable effort has been expended in industrial efforts to develop improved orally bioavailable selective estrogen receptor degraders (SERDs), improvements in clinical response have been incremental at best as compared to other approved endocrine therapies or to the only SERD currently FDA approved, fulvestrant. Brain penetration may be a differentiator between these SERDs in development, although the potential benefit for degrading estrogen receptor within the brain is currently unknown. However, estrogen receptor regulated pathways within the brain are known to impact metabolism and bone, both of which are important in immune function. To best deploy SERDs in the clinical treatment of breast and other estrogen responsive cancers, an understanding of the beneficial and/or detrimental effects of the estrogen receptor and its targeting within the brain is needed.

### SUMMARY

In an aspect, the disclosure relates to a method of treating cancer in a subject. The method may include administering to the subject at least one brain-excluded estrogen receptor (ER) modulating drug such that the drug exposure in the brain is zero to minimal. In some embodiments, the drug is administered at a dose and/or via a route such that the drug exposure in the brain is zero to minimal. In some embodiments, the at least one brain-excluded ER modulating drug is administered to the subject at a dose that results in from zero to minimal brain exposure. In some embodiments, the minimal brain exposure is 10:90 ratio drug concentration in CSF versus plasma. In some embodiments, the dose results in no more than a 10% decrease in FES-PET signal intracranially relative to a control. In some embodiments, the dose results in no more than a 20% decrease in FES-PET signal intracranially relative to a control. In some embodiments, ER expression in the brain is not reduced relative to a control.

In a further aspect, the disclosure relates to a method of inhibiting an estrogen receptor (ER) peripherally in a subject but not in the brain. The method may include administering to the subject at least one brain-excluded estrogen receptor (ER) modulating drug such that the drug exposure in the brain is zero to minimal. In some embodiments, the drug is administered at a dose and/or via a route such that the drug exposure in the brain is zero to minimal. In some embodiments, the at least one brain-excluded ER modulating drug is administered to the subject at a dose that results in from zero to minimal brain exposure. In some embodiments, the minimal brain exposure is 10:90 ratio drug concentration in CSF versus plasma. In some embodiments, the dose results in no more than a 10% decrease in FES-PET signal intracranially relative to a control. In some embodiments, the dose results in no more than a 20% decrease in FES-PET signal intracranially relative to a control. In some embodiments, ER expression in the brain is not reduced relative to a control.

In some embodiments, the method further includes administering to the subject at least one brain-penetrating ER agonist or at least one brain-penetrating androgen receptor (AR) modulator. In some embodiments, hot flashes in the subject are reduced. In some embodiments, the duration, and/or intensity, and/or frequency of the hot flashes are reduced. In some embodiments, the at least one brain-penetrating ER agonist comprises estrogen, estradiol, estetrol, estrone, conjugated estrogen, 10β,17β-dihydroxyestra-1,4-dien-3-one, 10β,17α-Dihydroxyestra-1,4-dien-3-one, or a combination thereof. In some embodiments, the at least one brain-excluded ER modulating drug and the at least one brain-penetrating ER agonist are administered simultaneously or sequentially. In some embodiments, the at least one brain-penetrating AR modulator comprises testosterone, a selective AR modulator, DHEA, or androstendiol, or a combination thereof. In some embodiments, the at least one brain-excluded ER modulating drug and the at least one brain-penetrating AR modulator are administered simultaneously or sequentially. In some embodiments, the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof. In some embodiments, the SERM is selected from lasofoxifene, bazodoxifene, tamoxifen, raloxifene, clomiphene, ospemiphene, arzoxifene, toremifene, and H3B6545, or a combination thereof. In some embodiments, the SERD is selected from camizestrant, fulvestrant, LSZ102, LY3484356, giredestrant, GDC0927, D-052, AC0682, AZD9496, SAR439859, RAD1901, G1T48, Zn-c5, ARV-471, and OP-1250, or a combination thereof. In some embodiments, the at least one brain-excluded ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months. In some embodiments, the at least one brain-excluded ER modulating drug is administered to the subject for 1 year, 2 years, 3 years, 4years, 5 years, or more than 5 years. In some embodiments, the at least one brain-excluded ER modulating drug is administered to the subject orally, intravenously, transdermally, nasally, or vaginally. In some embodiments, the ER is ER-alpha or ER-beta. In some embodiments, the cancer is selected from breast cancer, melanoma, colon cancer, glioblastoma, ovarian cancer, head cancer, neck cancer, and lung cancer, or a combination thereof.

In a further aspect, the disclosure relates to composition for treating cancer. The composition may include at least one brain-excluded estrogen receptor (ER) modulating drug and at least one brain-penetrating ER agonist. In a further aspect, the disclosure relates to a composition for treating cancer. The composition may include at least one brain-excluded estrogen receptor (ER) modulating drug and at least one brain-penetrating androgen receptor (AR) modulator. In some embodiments, the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof.

In some embodiments, the SERM is selected from lasofoxifene, bazodoxifene, tamoxifen, raloxifene, clomiphene, ospemiphene, arzoxifene, toremifene, and H3B6545, or a combination thereof. In some embodiments, the SERD is selected from fulvestrant, LSZ102, LY3484356, giredestrant, camizestrant, GDC0927, D-052, AC0682, AZD9496, SAR439859, RAD1901, G1T48, Zn-c5, ARV-471, and OP-1250, or a combination thereof. In some embodiments, the at least one brain-penetrating ER agonist comprises estrogen, estradiol, estetrol, estrone, conjugated estrogen, 10β,17β-dihydroxyestra-1,4-dien-3-one, 10β,17α-Dihydroxyestra-1,4-dien-3-one, or a combination thereof. In some embodiments, the at least one brain-penetrating AR modulator comprises testosterone, a selective AR modulator, DHEA, or androstendiol. In some embodiments, the cancer is selected from breast cancer, melanoma, colon cancer, glioblastoma, ovarian cancer, head cancer, neck cancer, and lung cancer, or a combination thereof.

The disclosure provides for other aspects and embodiments that will be apparent in light of the following detailed description and accompanying figures.

### BRIEF DESCRIPTION OF THE DRAWINGS

FIG. 1. Specific ablation of ERα expression in the brain results in E2-independent growth of tumors. Shown in FIG. 1 are graphs of tumor growth with placebo or estradiol (E2) treatment. Using a cre/lox transgenic approach, estrogen receptor alpha expression was ablated only in the brain in transgenic mice expressing the Cre enzyme. Cre+ mice and Cre− littermate control mice (LM) were ovariectomized and then received estrogen (E2) or placebo (P) treatment via drinking water. Estrogen responsive murine melanoma (BPD6) or mammary (E0771 or A7C11) cancer cells were engrafted orthotopically and tumor growth was monitored. Presented in FIG. 1 is a graph of the average tumor growth observed in Cre+ or LM mice receiving E2 or placebo treatment for each of these 3 tumor models. In each experiment, E2 treatment accelerated the growth of the tumors in the LM mice as compared to the placebo treated LM control, while tumors grew in the Cre+ placebo treated mice at a rate comparable to that observed for the E2 treated LM control mice. Thus, ablation of ER expression in the brain resulted in accelerated tumor growth reminiscent of E2 induction of tumor growth.

FIGS. 2A-2B. Brain penetration is an important distinguishing feature of SERDs. To determine whether the tumor growth phenotype observed in FIG. 1 could be recapitulated pharmacologically, BPD6 tumors were implanted into wild-type mice that had been ovariectomized and then assigned to E2 or placebo treatment. These groups were further subdivided to treatment with a brain penetrant (3964) or brain excluded (3574) SERD. As shown in FIG. 2A, estrogen treatment of these mice resulted in an approximate 10-fold increase in uterine wet weight (blue vs. red). Treatment with placebo together with either SERD alone (left side) did not affect uterine weight. Thus, neither SERD exhibited ER agonist activity in this sensitive measure of estrogenic activity. Administration of either SERD also reversed E2 stimulation of uterine wet weight, demonstrating that in the periphery these SERDs behave comparably as ER antagonists. FIG. 2B shows the tumor growth observed in the placebo treated arm. As compared to placebo/vehicle treatment, administration of E2 increased tumor growth by 3-fold (blue vs red). While the brain excluded SERD 3574 alone was without effect on tumor growth, administration of the brain penetrant SERD 3964 resulted in a 3-fold induction of tumor growth, equivalent to that observed for E2 treatment and reminiscent of the phenotype observed for the brain-ERKO mice (FIG. 1).

FIGS. 3A-3B. Brain penetration is a key distinguishing feature of SERDs (melanoma model). FIGS. 3A-3B are graphs of tumor growth with a peripheral SERD or a brain penetrating SERD, with or without estradiol, and the tumor growth data for the estrogen treated arm of the experiment described in FIGS. 2A-2B. Some curves of treatment groups were in common with those in FIG. 2B. To facilitate interpretation, the curves for placebo and E2 treatment arms were separated. As shown in FIG. 3A, while treatment with the brain excluded SERD 3574 did not itself affect tumor growth in the placebo treated animals, treatment with 3574 not only reversed E2 induced tumor growth, but in fact significantly repressed tumor growth as compared to placebo treatment alone. As shown in FIG. 3B, while treatment of E2-treated mice with a low dose (1 mg/kg) of the brain penetrant SERD 3964 reversed E2 stimulation of tumor growth, a high dose (10 mg/kg) of this SERD was less effective in tumor growth inhibition. This data was reminiscent of the clinical data observed in breast cancer treatment trials of SERDs known to access the brain, in which lower doses were more effective than higher doses.

FIGS. 4A-4B. The inverse dose response noted is reminiscent of that observed in recent clinical trials. FIGS. 4A-4B are graphs of tumor growth or uterotropic activity. E0771 mammary tumors were implanted orthotopically in ovariectomized mice receiving placebo or E2 treatment. These were further randomized to treatment with vehicle or SERDs 3574 or 3964. As shown in FIG. 4A, E2 treatment alone resulted in approximately 2-fold greater tumor volume than the placebo control (blue vs red). As shown in FIG. 4B, treatment with brain excluded SERD 3574 reversed E2 stimulation of tumor growth, whereas the response to brain penetrant SERD 3964 was more complex. A low dose of 3964 (1 mg/kg) reversed E2 stimulation of tumor growth, whereas treatment with a high dose (10 mg/kg) of 3964 was without effect on E2 induced tumor growth. Analysis of post-mortem uterine wet weight in these animals revealed that all doses of either SERD reversed E2 stimulation of the uterus; thus, the lack of effect of high dose 3964 on tumor growth was not due to (a) latent ER agonist activity or (b) inability to reverse E2 signaling in the periphery.

These data were also reminiscent of those observed in clinical trials of brain penetrant SERDs in breast cancer treatment, in which lower doses proved to be more effective in delaying progression of tumors.

FIG. 5. TUMOR GROWTH IS REPRESSED WITH ESTROGEN AND A BRAIN-EXCLUDED SERD. FIG. 5 is a graph of tumor growth for BPD6 tumors with or without ER for the various indicated drug treatments. Pro-tumor effects of estrogens on tumor associated macrophages have been shown to underly some of the tumor promoting effects of E2. An analogous experiment to that presented in FIGS. 2A-2B and FIGS. 3A-3B was conducted in mice in which ER expression in macrophages was ablated using a promoter (LysM) to drive expression of the Cre enzyme to delete this receptor specifically in this cell type in mice having a floxed allele of ER. BPD6 melanoma tumors were implanted subcutaneously into Cre+ (ER ablated in macrophage) or littermate (LM, phenotypically wild type) mice that had been ovariectomized and then assigned to placebo or E2 treatment. As shown in FIG. 5, whereas E2 treatment resulted in a 2-fold greater tumor volume in Cre− LM control mice (orange vs black), as compared to the placebo control, E2 was without significant effect on tumor growth in the Cre+ mice (blue vs green). While treatment with the brain excluded 3574 SERD did not affect the growth of tumors in placebo treated Cre+ mice, treatment with 3574 and E2 together significantly inhibited tumor growth as compared to the Cre+ mice receiving E2 treatment alone (green vs purple). Thus, the tumor suppressing activities of the brain excluded SERD that were revealed only when co-administered with E2 were independent of the previously known tumor promoting effects of estrogens through ER activation in macrophages.

FIG. 6. Fulvestrant, an approved SERD that also enters the brain, also promotes tumor growth in a dose related manner. FIG. 6 is a graph of tumor growth in ER-negative BPD6 melanoma cells with various treatments. BPD6 tumors were implanted orthotopically into ovariectomized mice receiving placebo or E2 treatment. These mice were further assigned to treatment with 1 of 3 doses of fulvestrant (25, 50, or 100 mg/kg). As shown, all of these doses reversed E2 stimulation of tumor growth. However, in placebo treated mice, only the highest (100 mg/kg) dose, which was the dose most likely to result in brain exposure and ER turnover in the brain, induced tumor growth itself (as compared to the placebo treated control). These data may shed light on the only moderate improvement in clinical response that was observed upon escalation of the approved dose of fulvestrant for breast cancer treatment from 250 mg to 500 mg.

FIG. 7. SERM/SERD pharmacology is more complex than expected. FIG. 7 is a model of how peripheral and brain-penetrating SERMs/SERDs impact tumor growth. The data presented herein support a tumor suppressing role for ER in the brain as well as a tumor supporting role for ER in the periphery and/or in the tumor microenvironment. ER activation (by E2 or by an appropriate SERM) in the brain can engage this tumor suppressive role, and further treatment with an ER antagonist/SERD that is restricted to the periphery can inhibit the tumor promoting effects of estrogens. Conversely, treatment with a brain penetrant SERD can result in turnover of ER in the brain, thereby negating the tumor suppressive signaling from brain to tumor and resulting in either lack of or diminished efficacy in tumor growth inhibition.

FIGS. 8A-8B. SERDs differ in the efficacy of ER downregulation in breast cancer cell lines. Breast cancer cells, including MCF7, BT483, T47D, ZR751, and CAMA1, were plated in phenol red free media supplemented with charcoal stripped FBS prior to treatment in triplicate wells with the indicated SERMs or SERDs (10-11-10-5 M final concentration). 24 hours after treatment, cells were fixed to the plates, and ER levels were analyzed by in-cell western using a fluorophore conjugated secondary antibody. Average detected ER levels were normalized to well DNA content using DRAQ5 staining. Relative ER turnover was calculated as a ratio of normalized ER levels detected in treated wells to untreated wells. Data were representative of at least 3 independent experiments. Data for each cell line were graphed in 2 panels to categorize SERDs by chemical properties, separating basic SERDs (FIG. 8A) and those having carboxylic acid group(s) (FIG. 8B). Serm-like SERDs bazedoxifene and elacestrant were also presented in (FIG. 8A) for reference. Fulvestrant was included on both graphs for all cell lines to facilitate comparison.

FIGS. 9A-9C. Anti-proliferative activities of SERDs in select breast cancer cell lines differ primarily in potency. Breast cancer cells were plated in phenol red free media supplemented with charcoal stripped FBS prior to treatment in triplicate wells with the indicated SERMs and SERM-like SERDs (FIG. 9A), acidic SERDs (FIG. 9B), basic SERDs (FIG. 9C) at 10-12 to 10-5 M final concentration, followed by addition of estradiol (E2, 0.1 nM) or insulin (2 nM, MCF7 cell line only, bottom panels). 7 days after treatment, plates were harvested and DNA quantitated using fluorescence following Hoechst staining. Average detected fluorescence was normalized to wells receiving no ligand treatment, and relative change in DNA fluorescence was calculated compared to wells treated only with E2. Data are representative of at least 3 independent experiments.

FIG. 10. Relationship between ER turn over and ER inhibition, The data in FIGS. 8A-8B and FIGS. 9A-9C was used to plot % ER turnover vs percent inhibition of estrogen dependent proliferation using the average value derived from 3 independent experiments and utilizing the data recorded for 10-7 M treatments only. Indicated are points corresponding to treatment with a hypothetical “ideal” SERD (100% degradation/100% inhibition, green, circled), elacestrant (red, circled) and ZN-c5 (blue, AZD9833, circled).

FIGS. 11a-11b. Fulvestrant Inhibits Estrogen Induced Tumor Growth, but itself can induce tumor growth. As shown in FIG. 11A, A7C11 tumors were mammary tumors were orthotopically grafted into C57BL6 mice receiving placebo or estradiol (2.72 mog/mL in drinking water) treatment. 2 days later mice were subdivided to receive treatment with vehicle or fulvestrant (25, 50 or 100 mg/kg sc q5d). Average tumor volume throughout treatment (FIG. 11A) and uterine weight (FIG. 11B) at necropsy are presented. Placebo and E2+Veh groups in (FIG. 11A) are common between graphs to facilitate comparison. * p<0.05, 2-way (FIG. 11A) or 1-way (FIG. 11B) ANOVA followed by Tukey multiple comparison test. As shown in FIG. 11B, profiling of tumor infiltrating immune cells showed significant increases in G-MDSCs present in a subset of groups as indicated.

FIGS. 12A-12D. Brain excluded and penetrant SERDs differ in their activities in mammary cancer tumors. Shown in FIG. 12A are graphs of uterine weight for placebo and estrogen treated mice, and ER expression in the uterus and liver after various treatments. A7C11 tumors were mammary tumors were orthotopically grafted into C57BL6 mice receiving placebo or estradiol (2.72 mcg/mL in drinking water) treatment. 2 days later, mice were subdivided to receive treatment with vehicle or brain excluded SERD 3574 (FIG. 12B), brain penetrant SERDs 3964 (FIG. 12C), or elacestrant (FIG. 12D), respectively.

Average tumor volume throughout treatment and uterine weight at necropsy are presented. * p<0.005, 2-way ANOVA followed by Tukey multiple comparison test. Uterine weights recorded at necropsy are presented in corresponding colors to the right of each study.

FIGS. 13A-13B. Brain excluded and penetrant SERDs differ in their activities in mammary cancer tumors. As shown in FIG. 13A, E0771 tumors were mammary tumors were orthotopically grafted into C57BL6 mice receiving placebo (FIG. 13A) or estradiol (2.72 mcg/mL in drinking water) (FIG. 13B) treatment. 2 days later mice were subdivided to receive treatment with vehicle or brain excluded or penetrant SERDs KP-3574 (10 mg/kg) or KP-3964 (1 or 10 mg/kg). Average tumor volume throughout treatment (FIG. 13A) and uterine weight (FIG. 13B) at necropsy are presented. * p<0.005, 2-way ANOVA (A) or 1-way ANOVA followed by Tukey multiple comparison test. As shown in FIG. 13B, brain excluded and penetrant SERDs differed in their activities in mammary cancer tumors. E0771 tumors were mammary tumors were orthotopically grafted into C57BL6 mice receiving placebo (FIG. 13A) or estradiol (2.72 mcg/mL in drinking water) (FIG. 13B) treatment. 2 days later mice were subdivided to receive treatment with vehicle or brain excluded or penetrant SERDs KP-3574 (10 mg/kg) or KP-3964 (1 or 10 mg/kg). Immune profiling of tumor infiltrating immune cells are indicated, including the ratio between M1/M2 macrophages (CD45+, ETC) and G-MDSC (CD45+, ETC). * p<0.005, 2-way ANOVA (FIG. 13A) or 1-way ANOVA (FIG. 13B) followed by Tukey multiple comparison test.

FIG. 14. Brain excluded SERM Lasofoxifene cooperates with E2 to repress the growth of breast cancer tumor models. A7C11 mammary tumors were orthotopically grafted into C57BL6 mice receiving placebo or estradiol (2.72 mcg/mL in drinking water) treatment. 2 days later mice were subdivided to receive treatment with vehicle or Lasofoxifene (1 or 3 mg/kg po qd, orally daily). Average tumor volume throughout treatment and uterine weight at necropsy are presented. * p<0.005, 2-way ANOVA followed by Tukey multiple comparison test.

### DETAILED DESCRIPTION

Described herein are compositions and methods for treating cancer, such as melanoma, lung cancer, and breast cancer. The compositions and methods may include at least one estrogen receptor (ER) modulating drug. The compositions and methods may further include at least one brain-penetrating ER agonist and/or at least one brain-penetrating androgen receptor (AR) modulator.

In studying the role of estrogens in modulating immune cell activity in the tumor microenvironment, the inventors have discovered that in syngeneic murine cancer models in which estrogen treatment stimulates tumor growth (including models of mammary, lung, and melanoma cancers), estrogen treatment generates an immune suppressive environment.

This is accomplished through estrogen promoting the skewing of the phenotype of tumor infiltrating macrophages toward an anti-inflammatory M2-like character and away from the inflammatory M1-like phenotype.

In evaluating the role of ER expression and action in the brain with respect to its effects on tumor growth in the periphery, the inventors have determined with transgenic mouse models that deletion of ER in the brain alters the growth kinetics of estrogen stimulated tumors. Specifically, loss of ER in the brain accelerates the growth of tumors in the periphery such that, in the absence of estrogen treatment, tumor growth occurs that is equivalent to that observed in the estrogen tumors grown in wild-type mice. These data suggest a brain-to-tumor signaling pathway that represses tumor growth in an ER dependent manner.

As detailed herein, selective estrogen receptor degrader (SERD) compounds that are either (a) excluded from the brain or (b) highly brain penetrant have been examined. The inventors have discovered that, given the data with brain specific deletion of ER, treatment with a brain penetrant SERD resulted in accelerated tumor growth in the absence of estrogen treatment, and it also compromised the ability of the brain penetrant SERD to oppose estrogen stimulated tumor growth.

One surprising and highly unanticipated result was also noted. It was surprisingly found that treatment with estrogen together with the brain excluded SERD resulted in tumor growth significantly suppressed as compared to the untreated placebo (no estrogen control). Thus, inhibition of estrogen action in the periphery (by the SERD acting on tumor cells, immune cells, or other components of the tumor microenvironment) coupled with estrogen exposure in the brain may be used to treat estrogen responsive cancers.

The compositions and methods detailed herein include a combined treatment regimen for the treatment of estrogen responsive cancers (such as breast, lung, melanoma, or other cancers) in which estrogens (by any route/formulation) are administered concurrent with treatment with a selective estrogen receptor degrader (for example, fulvestrant, elacestrant, Camizestrant, or other drugs of similar activity), or a selective estrogen receptor modulator (for example, bazedoxifene, lasofoxifene, or other like compounds) or another anti-estrogenic therapy, including an ER-directed PROTAC (for example, ARV-471 or other like compounds).

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%; 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art.

Alternatively, such as with respect to biological systems or processes, the term “about” can mean within an order of magnitude, preferably within 5-fold, and more preferably within 2-fold, of a value.

The term “administration” or “administering,” as used herein refers to providing, contacting, and/or delivery an agent or composition as detailed herein, by any appropriate route to achieve the desired effect. These agents may be administered to a subject in numerous ways and may be used in combination.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Antiprogestogens” and “antiprogestins” as used herein, are used interchangeably and refer to the class of drugs/compounds that act as progesterone antagonists or progesterone blockers and prevent progestogens (for example, progesterone) from mediating their biological effects in the body of a subject.

“Aromatase inhibitor” as used herein refers to the class of compounds/drugs that target aromatase, which is an enzyme involved in the biosynthesis of estrogen. Aromatase inhibitors may block the production of estrogen or block the action of estrogen on receptors.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Cancer” refers to all types of cancer or neoplasm or malignant tumors found in animals, including carcinoma, adenoma, melanoma, sarcoma, lymphoma, leukemia, blastoma, glioma, astrocytoma, mesothelioma, or a germ cell tumor. Cancer may include cancer of, for example, the colon, rectum, stomach, pancreas, bladder, cervix, uterus, vulva, endometrium, salivary gland, skin, epithelium, muscle, kidney, liver, lymph, thyroid, bone, blood, ovary, prostate, lung, brain, head, neck, head and neck, and/or breast. Cancer may include medulloblastoma, non-small cell lung cancer, small cell lung cancer, gastrointestinal, neuroblastoma, glioblastoma, peripheral neuroepithelioma, hepatoma, colorectal cancer, uterine cervical cancer, melanoma, myeloma, and/or mesothelioma. The cancer may include leukemia. The cancer may include any metastasis of the cancer. The term “leukemia” refers to broadly progressive, malignant diseases of the hematopoietic organs/systems and is generally characterized by a distorted proliferation and development of leukocytes and their precursors in the blood and bone marrow. Leukemia diseases include, for example, chronic myeloid leukemia (CML), acute myeloid leukemia (AML), acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leucocythemia leukemia, basophilic leukemia, blast cell leukemia, bovine leukemia, chronic myelocytic leukemia, leukemia cutis, embryonal leukemia, eosinophilic leukemia, Gross'leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid leukemia, myeloid granulocytic leukemia, myelomonocytic leukemia, Naegeli leukemia, plasma cell leukemia, plasmacytic leukemia, and promyelocytic leukemia. In some embodiments, the cancer is selected from melanoma, lung cancer, breast cancer, glioblastoma, ovarian cancer, and colon cancer, and metastatic variations thereof. In some embodiments, the cancer comprises melanoma. In some embodiments, the cancer comprises breast cancer.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P.J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity or for another parameter may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Effective amount” or “therapeutically effective amount” refers to an amount sufficient to effect beneficial or desirable biological and/or clinical results.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous.

Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Selective Estrogen Receptor Degrader or Downregulator” or “SERDs” are used interchangeably and refer to those class of drugs/compounds that bind to the estrogen receptor (ER) and, in the process of doing so, causes the estrogen receptor to be degraded and thus downregulated.

“Selective Estrogen Receptor Modulators” or “SERMs” refers to the class of drugs/compounds that act on the estrogen receptor (ER).

“Subject” and “patient” as used herein interchangeably refers to any vertebrate, including, but not limited to, a mammal that wants or is in need of the herein described compositions or methods. The methods and compositions disclosed herein can be used on a sample either in vitro (for example, on isolated cells or tissues) or in vivo in a subject (for example, a living organism, such as a patient). The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment. In some embodiments, the subject has cancer. In some embodiments, the subject is in remission from cancer.

“Substantially identical” can mean that a first and second amino acid or polynucleotide sequence are at least 60%, 65%, 70%, 75%, 80%, 85%, 90%, 95%, 96%, 97%, 98%, or 99%, or less than 100% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91 %, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91 %, 92%, 93%, 94%, 95%, 96%, 97%, 98%, or 99%, or less than 100% identical over the full length of the amino acid sequence or a fragment thereof.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Estrogen Receptor (er) Modulating Drug

Provided herein are estrogen receptor (ER) modulating drugs, which may also be referred to as an “ER modulator.” The term “estrogen receptor (ER) modulating drug” refers to any drug/compound, or class of drug/compound that is capable of modulating the estrogen receptor in/on a cell. ER receptors may include, for example, canonical ERs (for example, nuclear ER) and the atypical GPER at the membrane. The ER may be ER-alpha, or ER-beta, or a combination thereof. Modulating may include inhibiting. An ER modulating drug may be, for example, an ER inhibitor or an ER antagonist. An ER modulating drug may bind an estrogen receptor. An ER modulating drug may prevent or reduce the binding of a molecule to the estrogen receptor. An ER modulating drug may increase and/or prolong the binding of a molecule to the estrogen receptor. An ER modulating drug may decrease or reduce the activity of the estrogen receptor. An ER modulating drug may increase or enhance the activity of the estrogen receptor.

The ER modulating drug may be a brain-excluded ER modulating drug. Administration of the brain-excluded ER modulating drug may result in from zero to minimal brain exposure in a subject. The brain-excluded ER modulating drug may be administered at a dose and/or via a route such that the drug exposure in the brain, or in at least one region of the brain, is zero to minimal. Regions of the brain may include, for example, forebrain, midbrain, hindbrain, cerebellum, cerebrum, cortex, thalamus, hypothalamus, subthalamus, epithalamus, pituitary gland, third ventricle, hippocampus, basal ganglia, white matter, subcortical, rhinencephalon, cerebral cortex, globus pallidus (GPI), subthalamic nucleus (STN), ventral intermediate thalamus, subthalamic nucleus, internal globus pallidus, NKX neurons, and KNDY neurons such as KNDY neurons in the arcuate nucleus of the basal hypothalamus (brain-ERKO). In some embodiments, administration of the brain-excluded ER modulating drug results in from zero to minimal brain exposure in the hypothalamus of a subject. The dose for penetrating the brain may depend on the particular ER modulating drug. The level of brain exposure may be determined by any suitable means known in the art.

A brain-excluded ER modulating drug may be administered to a subject at a dose that results in from zero to minimal brain exposure. The dose of a brain-excluded ER modulating drug that results in from zero to minimal brain exposure in a subject may be determined or defined by determining the expression level of a gene. In some embodiments, the expression of the gene is increased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be increased by about 5-95%, 10-90%, 15-85%, 20-80%, or 1.5-fold to 10-fold, relative to a control. In some embodiments, the expression of the gene is decreased by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be decreased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene may be decreased by about 5-95%, 10-90%, 15-85%, 20-80%, or 1.5-fold to 10-fold, relative to a control. The level of expression of the gene in the subject, or in a sample therefrom, may be determined by any suitable means known in the art, which may include, for example, antibody binding, Western blot analyses, Northern blot hybridization analyses, hybridization of a probe to a gene transcript such as on a microarray, amplification-based detection methods such as reverse-transcription based polymerase chain reaction (RT-PCR) or quantitative RT-PCR or RNA sequencing, or a combination thereof. The expression level of the genes can be analyzed based on the biological activity or quantity of proteins encoded by the genes. The gene expression levels may be determined by measuring mRNA or protein levels of the genes. The protein levels of a biomarker may be determined using proteomics, immunoassay, enzyme-linked immunoassay (ELISA), radioimmunoassay (RIA), a competitive inhibition assay such as forward or reverse competitive inhibition assays, a fluorescence polarization assay, a competitive binding assay, or a combination thereof.

In some embodiments, the minimal brain exposure is 10:90 ratio drug concentration in cerebrospinal fluid (CSF) versus plasma. In some embodiments, the dose results in no more than a 10% decrease in F-18,16 alpha-fluoroestradiol (FES) positron emission tomography (PET)/computed tomography (CT) (FES-PET) signal intracranially relative to a control, to be classified as zero to minimal brain exposure in a subject. In some embodiments, the dose results in no more than a 20% decrease in FES-PET signal intracranially relative to a control, to be classified as zero to minimal brain exposure in a subject. In some embodiments, administration of the brain-excluded ER modulating drug does not reduce ER expression in the brain, relative to a control.

An ER modulating drug may modulate an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%; 1.5-fold, 2-fold, 3-fold, 4-fold, 5-fold, 6-fold, 7-fold, 8-fold, 9-fold, or 10-fold, relative to a control. An ER modulating drug may modulate an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may modulate an ER receptor by about 5-95%, 10-90%, 15-85%, 20-80%, or 1.5-fold to 10-fold, relative to a control.

An ER modulating drug may have agonist activity against an ER receptor. An ER modulating drug may increase or enhance the activity of an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may increase or enhance the activity of an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may increase or enhance the activity of an ER receptor by about 5-95%, 10-90%, 15-85%, 20-80%, or 1.5-fold to 10-fold, relative to a control.

An ER modulating drug may have antagonist activity against an ER receptor. An ER modulating drug may decrease or inhibit the activity of an ER receptor by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%; 1.5-fold, 2-fold, 3-fold, 4-fold, 5-fold, 6-fold, 7-fold, 8-fold, 9-fold, or 10-fold, relative to a control. An ER modulating drug may decrease or inhibit the activity of an ER receptor by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. An ER modulating drug may decrease or inhibit the activity of an ER receptor by about 5-95%, 10-90%, 15-85%, 20-80%, or 1.5-fold to 10-fold, relative to a control.

ER modulating drugs may comprise a small molecule, peptide, polypeptide, antibody, nucleotide, polynucleotide, lipid, or carbohydrate, or a combination thereof. ER modulating drugs may include, for example, a selective estrogen receptor modulator (SERM), a selective estrogen receptor degrader (SERD), Complete Estrogen Receptor Antagonist (CERAN), an aromatase inhibitor, a gonadotropin-releasing hormone agonist (GNRH) agonist, or a combination thereof. An effective amount of the ER modulating drug may be administered.

a. Serm

SERMs may comprise a small molecule, peptide, polypeptide, antibody, nucleotide, polynucleotide, lipid, or carbohydrate, or a combination thereof. SERMs may be synthesized and/or extracted and/or purified by any suitable means known in the art. SERMs may be commercially available. SERMs may include, for example, lasofoxifene (FABLYN®), bazodoxifene, tamoxifen (NOLVADEX®; TAMIFEN®), raloxifene (EVISTA®), toremifene (FARESTON®), or arzoxifene (also known as LY-353381), ospemifene (OSPHENA®; SENSHIO®), clomiphene (also known as clomiphene; CLOMID®; SEROPHENE®), or H3B6545, or a combination thereof. Examples of other SERMS are described in International Patent Application No. PCT/US2015/023216 published as WO 2015/149045, U.S. Pat. No. 7,612,114, U.S. Pat. No. 7,960,412, U.S. Pat. No. 8,399,520, U.S. Patent Publication No. US 2009-0325930, and U.S. Patent Publication No. US 2006-0116364, the contents of which are incorporated by reference in their entirety. In some embodiments, the ER modulating drug comprises lasofoxifene.

b. Serd

SERDs may comprise a small molecule, peptide, polypeptide, antibody, nucleotide, polynucleotide, lipid, or carbohydrate, or a combination thereof. SERDs may be synthesized and/or extracted and/or purified by any suitable means known in the art. SERDs may be commercially available. SERDs may include, for example, ICI 182780 (also known as fulvestrant; FASLODEX®), LSZ102, LY3484356, giredestrant (also known as GDC9545), camizestrant (also known as AZD-9833 or 3574), AZD9496, GDC0927, D-052, AC0682, SAR439859 (also known as amcenestrant), RAD1901 (also known as elacestrant), G1T48 (also known as rintodestrant), Zn-c5, ARV-471, or OP-1250, or a combination thereof. In some embodiments, the ER modulating drug comprises camizestrant.

c. Complete Estrogen Receptor Antagonist (CERAN)

Complete Estrogen Receptor Antagonists (CERAN) may comprise a small molecule, peptide, polypeptide, antibody, nucleotide, polynucleotide, lipid, or carbohydrate, or a combination thereof. “CERAN” refers to a molecule that is an antagonist of the estrogen receptor alpha (ER-alpha; ERa; ESR1; nuclear receptor subfamily 3, group A, member 1; NR3A1) and a selective ER degrader (SERD), with potential antineoplastic activity. A CERAN may be orally available. A CERAN may be a small molecule. For example, a CERAN may be an estrogen receptor antagonist that precludes binding of another ER ligand, and (in and of itself) exhibits no partial agonist activity in any estrogen responsive tissue. CERANs may be synthesized and/or extracted and/or purified by any suitable means known in the art. CERANs may be commercially available. One example of a CERAN is OP-1250.

d. Gonadotropin-Releasing Hormone (GNRH) Agonist

Gonadotropin-releasing Hormone (GNRH) agonists affect gonadotropins and sex hormones. They are agonists of the GnRH receptor and work by increasing or decreasing the release of gonadotropins and the production of sex hormones by the gonads. GNRH agonists may comprise a small molecule, peptide, polypeptide, antibody, nucleotide, polynucleotide, lipid, or carbohydrate, or a combination thereof. GNRH agonists may be synthesized and/or extracted and/or purified by any suitable means known in the art. GNRH agonists may be commercially available. GNRH agonists may include, for example, buserelin, gonadorelin, goserelin, histrelin, leuprorelin, nafarelin, and triptorelin, or a combination thereof.

e. Aromatase Inhibitor

Aromatase inhibitors may comprise a small molecule, peptide, polypeptide, antibody, nucleotide, polynucleotide, lipid, or carbohydrate, or a combination thereof. Aromatase inhibitors may be synthesized and/or extracted and/or purified by any suitable means known in the art. Aromatase inhibitors may be commercially available. Aromatase inhibitors may include, for example, letrozole (FEMARA®), anastrozole (ARIMIDEX®), Exemestane (AROMASIN®), vorozole, formestane (LENTARON®), fadrozole (AFEMA®), testolactone (TESLAC®), aminoglutethimide (ELIPTEN®; CYTADREN®; ORIMETEN®), androstatrienedione, or 4-androstene-3,6,17-trione (also known as 4-AT; 6-Oxo, 6-OXO ™M), or a combination thereof.

### 3. Brain-Penetrating Estrogen Receptor (ER) Agonist

A brain-penetrating estrogen receptor (ER) agonist may be administered with the brain-excluded ER modulating drug. A brain-penetrating ER agonist may activate or increase or enhance an activity of the ER. A brain-penetrating ER agonist may prevent or reduce the binding of an inhibitory molecule to the ER. A brain-penetrating ER agonist may increase and/or prolong the binding of a molecule to the ER. Administration of the brain-penetrating ER agonist may result in more than minimal brain exposure in a subject.

Brain-penetrating ER agonists may comprise a small molecule, peptide, polypeptide, antibody, nucleotide, polynucleotide, lipid, or carbohydrate, or a combination thereof. Brain-penetrating ER agonists may be synthesized and/or extracted and/or purified by any suitable means known in the art. Brain-penetrating ER agonists may be commercially available. Brain-penetrating ER agonist may include, for example, estrogen, estradiol, estetrol, estrone, conjugated estrogen, 10β,17β-dihydroxyestra-1,4-dien-3-one, or 10β,17α-Dihydroxyestra-1,4-dien-3-one, or a combination thereof.

The ER agonist may reduce or inhibit hot flashes in a subject. Upon administration of the ER agonist, the duration, and/or intensity, and/or frequency of hot flashes may be reduced. The ER agonist may reduce or inhibit hot flashes by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The ER agonist may reduce or inhibit hot flashes by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The ER agonist may reduce or inhibit hot flashes by about 5-95%, 10-90%, 15-85%, 20-80%, or 1.5-fold to 10-fold, relative to a control. The control may be a subject without treatment with an ER modulating drug. The amelioration of hot flashes may increase patient compliance with a cancer therapy such as an ER modulating drug. The amelioration of hot flashes may increase patient compliance with a cancer therapy such as an ER modulating drug relative to a therapy with an ER modulating drug but without an ER agonist.

The ER agonist may increase the effectiveness of an ER modulating drug in treating or reducing cancer. The ER agonist may cross the blood-brain barrier. The ER agonist may activate an ER receptor in the brain.

### 4. Brain-Penetrating Androgen Receptor (AR) Modulator

A brain-penetrating androgen receptor (AR) modulator may be administered with the brain-excluded ER modulating drug. Brain-penetrating AR modulators may be agonists or antagonists of the AR receptor. Brain-penetrating AR modulators may be a mixture of both agonists and antagonists of the AR receptor. A brain-penetrating AR modulator may inhibit or decrease or reduce, or activate or increase or enhance, an activity of the AR receptor. A brain-penetrating AR modulator may prevent or reduce the binding of a molecule to the AR. A brain-penetrating AR modulator may increase and/or prolong the binding of a molecule to the AR. Administration of the brain-penetrating AR modulator may result in more than minimal brain exposure in a subject.

Brain-penetrating AR modulators may comprise a small molecule, peptide, polypeptide, antibody, nucleotide, polynucleotide, lipid, or carbohydrate, or a combination thereof. Brain-penetrating AR modulators may be synthesized and/or extracted and/or purified by any suitable means known in the art. Brain-penetrating AR modulators may be commercially available. Brain-penetrating AR modulators may include, for example, testosterone, a selective AR modulator, DHEA, or androstendiol.

### 5. Additional Therapies

In some embodiments, the at least one ER modulating drug is combined with at least one additional cancer therapy. As used herein, the term “standard of care treatment” or “additional therapy” or “additional treatment” are used interchangeably and refer to any other standard cancer treatments/additional cancer treatments that do not include ER modulating drugs. Additional cancer therapies may comprise a small molecule, peptide, polypeptide, antibody, nucleotide, polynucleotide, lipid, or carbohydrate, or a combination thereof. Additional cancer therapies may be synthesized and/or extracted and/or purified by any suitable means known in the art. Additional cancer therapies may be commercially available. Additional cancer therapies may include, for example, chemotherapy, immunotherapy, radiation therapy, hormone therapy, targeted drug therapy, cryoablation, and surgery, or a combination thereof. Hormone therapy, for example, may block hormone synthesis such as blocking estrogen synthesis. An effective amount of the additional therapy may be administered.

Chemotherapies may include, for example, an antimitotic agent, an alkylating agent, an antimetabolite, an antimicrotubule agent, a topoisomerase inhibitor, a cytotoxic agent, a cell cycle inhibitor, a growth factor inhibitor, a histone deacetylase (HDAC) inhibitor, or an inhibitor of a pathway that cross-talks with and activates ER transcriptional activity, or a combination thereof.

Alkylating agents may include, for example, cisplatin (PLATINOL®), oxaliplatin (ELOXATIN®), chlorambucil (LEUKERAN®), procarbazine (MATULANE®; NATULAN®), or carmustine (BICNU®), or a combination thereof. Antimetabolites may include, for example, methotrexate (also known as amethopterin), 5-fluorouracil, cytarabine (also known as cytosine arabinoside or ara-C; CYTOSAR®), or gemcitabine (GEMZAR®), or a combination thereof. Antimicrotubule agents may include, for example, vinblastine (VELBAN®; VELBE®), or paclitaxel (TAXOL®), or a combination thereof. Topoisomerase inhibitors may include, for example, etoposide (VEPESID®), or doxorubicin (ADRIAMYCIN®; MYOCET®), or a combination thereof. Cytotoxic agents may include, for example, bleomycin (BLENOXANE®). Growth factor inhibitors may include, for example, human epidermal growth factor receptor 2 (HER2) inhibitors. HER2 inhibitors include, for example, trastuzumab (HERCEPTIN®), deruxtecan, sacitizumab, and/or ado-trastuzumab emtansine (KADCYLA®). HDAC inhibitors may include, for example, vorinostat (ZOLINZA®), romidepsin (ISTODAX®), chidamide (also known as tucidinostat; EPIDAZA®; HIYASTA™), panobinostat (FARYDAK®), belinostat (also known as BELEODAQ® or PXD101), valproic acid (DEPAKOTE®; DEPAKENE®; STAVZOR®)), mocetinostat (also known as MGCD0103), abexinostat (also known as PCI-24781), entinostat (also known as SNDX-275 or MS-275), pracinostat (also known as SB939), resminostat (also known as 4SC-201 or RAS2410), givinostat (also known as gavinostat or ITF2357), quisinostat (also known as JNJ-26481585), kevetrin, CUDC-101, AR-42, tefinostat (also known as CHR-2845), nanatinostat (also known as CHR-3996), domatinostat (also known as 4SC-202), ivaltinostat (also known as CG-200745), rocilinostat (also known as ACY-1215), or sulforaphane, or a combination thereof. Inhibitors of a pathway that cross-talks with and activates ER transcriptional activity may include, for example, a phosphoinositide 3-kinase (PI3K) inhibitor, a heat shock protein 90(HSP 90 ) inhibitor, or a mammalian target of rapamycin (mTOR) inhibitor. mTOR inhibitors include, for example, everolimus (AFINITOR®; VOTUBIA®; ZORTRESS®). In some embodiments, the HDAC inhibitor comprises vorinostat (ZOLINZA®). In some embodiments, the HDAC inhibitor comprises romidepsin (ISTODAX®). In some embodiments, the entinostat is not administered with an HER2 inhibitor. In some embodiments, the HDAC inhibitor comprises entinostat with the proviso that the subject is not treated with a HER2 inhibitor.

Immunotherapies may include, for example, a checkpoint inhibitor, or denosumab (PROLIA®; XGEVA®), or a combination thereof. “Checkpoint inhibitor” or “immune checkpoint inhibitor” may also be referred to as an immune checkpoint blockade (ICB) therapy. Checkpoint inhibitors may comprise an antibody. Checkpoint inhibitors may include, for example, an antibody to programmed cell death protein 1 (PD1) (anti-PD 1), or an antibody to cytotoxic T-lymphocyte-associated protein 4 (CTLA4) (anti-CTLA4), or an antibody to programmed death-ligand 1(PDL 1 ) (anti-PDL 1), or DMXAA (sting agonist; also known as ASA404, vadimezan, or dimethylxanthone acetic acid) or a combination thereof. “Anti-PD1” refers to an antibody that binds PD1, “anti-CTLA4” refers to an antibody that binds CTLA4, and “anti-PDL1” refers to an antibody that binds PDL1. In some embodiments, the PD-1 antibody comprises pembrolizumab (KEYTRUDA®) or nivolumab (OPDIVOo®). In some embodiments, the CTLA-4 antibody comprises ipilimumab (YERVOY®).

Targeted drug therapies may include, for example, vemurafenib (ZELBORAF®), anti-EGFR targeted therapies (such as, for example, erlotinib (TARCEVA®), and/or gefitinib (IRESSA®)), a serotonin-norepinephrine reuptake inhibitor (SNRI; such as venlafaxine (EFFEXOR XR®)), a selective serotonin reuptake inhibitor (SSRI), or gabapentin (NEURONTIN®), or a combination thereof. Targeted drug therapies may include an inhibitor of cyclin-dependent kinases 4 and/or 6 (CDK4/6), also referred to as CDK4/6 inhibitors. CDK4/6 inhibitors may include, for example, palbociclib (IBRANCE®), ribociclib (KISQALI®), and abemaciclib (VERZENIO®). Targeted drug therapies may include an inhibitor of cyclin-dependent kinases 7 (CDK7), also referred to as CDK7 inhibitors. CDK7 inhibitors may include, for example, CT7001 (also referred to as samuraciclib).

In some embodiments, the at least one ER modulating drug is combined with anti-PD1, or anti-CTLA4, or vemurafenib (ZELBORAF®), or a combination thereof.

### 6. Pharmaceutical Compositions

Further provided herein are pharmaceutical compositions comprising the above-described ER modulating drug(s). The pharmaceutical composition may further include at least one brain-penetrating ER agonist and/or at least one brain-penetrating AR modulator and/or at least one at least one additional cancer therapy. In some embodiments, the pharmaceutical composition may comprise about 1 ng to about 10 mg of ER modulating drug, or about 1 ng to about 10 mg of ER modulating drug and brain-penetrating AR modulator, or about 1 ng to about 10 mg of ER modulating drug and brain-penetrating ER agonist, or about 1 ng to about 10 mg of ER modulating drug and additional cancer therapy, or about 1 ng to about 500 mg of ER modulating drug, or about 1 ng to about 500 mg of ER modulating drug and brain-penetrating AR modulator, or about 1 ng to about 500 mg of ER modulating drug and brain-penetrating ER agonist, or about 1 ng to about 500 mg of ER modulating drug and additional cancer therapy, or about 1 ng to about 1 g of ER modulating drug, or about 1 ng to about 1 g of ER modulating drug and brain-penetrating AR modulator, or about 1 ng to about 1 g of ER modulating drug and brain-penetrating ER agonist, or about 1 ng to about 1 g of ER modulating drug and additional cancer therapy. The ER modulating drug as detailed herein, with or without at least one brain-penetrating ER agonist and/or at least one brain-penetrating AR modulator and/or at least one at least one additional cancer therapy, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal oil, vegetable oil, mineral oil or synthetic oil. Physiological saline solution, dextrose or other saccharide solution or glycols such as ethylene glycol, propylene glycol or polyethylene glycol can also be included. For parenteral administration, the ER modulating drug will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. Pharmaceutical compositions for vaginal topical administration can be in the form of ointment, cream, gel or lotion. The pharmaceutical compositions for vaginal topical administration often include water, alcohol, animal oil, vegetable oil, mineral oil or synthetic oil. Hydrocarbon (paraffin), wool fat, beeswax, macrogols, emulsifying wax or cetrimide can also be included.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition at a concentration less than 6 mg/mL.

The ER modulating drug, with or without the at least one brain-penetrating ER agonist and/or at least one brain-penetrating AR modulator and/or at least one at least one additional cancer therapy, may be present or formulated as a pharmaceutically acceptable salt thereof, or a prodrug thereof. The term “pharmaceutically acceptable salt” refers to non-toxic pharmaceutically acceptable salts (see Gould, International Journal of Pharmaceutics1986, 33, 201-217; and Berge et al., Journal of Pharmaceutical Sciences 1977, 66, 1-19).

Other salts well known to those in the art may, however, be used. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

Embodiments also include prodrugs of the compounds disclosed herein. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, H. Bundgaard, Elsevier, 1985.

Some of the crystalline forms for the compounds may exist as polymorphs and as such are intended to be included in the present invention. In addition, some of the compounds may form solvates with water (i.e., hydrates) or common organic solvents, and such solvates are intended to be encompassed by some embodiments.

Where the processes for the preparation of the compounds as disclosed herein give rise to mixtures of stereoisomers, these isomers may be separated by conventional techniques such as preparative chromatography. The compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers, cis-trans isomers, and enantiomers thereof are encompassed by some embodiments.

In embodiments wherein the pharmaceutical composition comprises both the at least one ER modulating drug and the at least one brain-penetrating ER agonist, they may be present in the pharmaceutical composition in a variety of molar ratios. The molar ratio between the at least one ER modulating drug and the at least one brain-penetrating ER agonist may be 1:1, or 1:15, or from 5:1 to 1:10, or from 1:1 to 1:5. The molar ratio between the at least one ER modulating drug and the at least one brain-penetrating ER agonist may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one brain-penetrating ER agonist may be less than 20:1, less than 15:1, less than 10:1, less than 9:1, less than 8:1, less than 7:1, less than 6:1, less than 5:1, less than 4:1, less than 3:1, less than 2:1, or less than 1:1.

In embodiments wherein the pharmaceutical composition comprises both the at least one ER modulating drug and the at least one brain-penetrating AR modulator, they may be present in the pharmaceutical composition in a variety of molar ratios. The molar ratio between the at least one ER modulating drug and the at least one brain-penetrating AR modulator may be 1:1, or 1:15, or from 5:1 to 1:10, or from 1:1 to 1:5. The molar ratio between the at least one ER modulating drug and the at least one brain-penetrating AR modulator may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one brain-penetrating AR modulator therapy may be less than 20:1, less than 15:1, less than 10:1, less than 9:1, less than 8:1, less than 7:1, less than 6:1, less than 5:1, less than 4:1, less than 3:1, less than 2:1, or less than 1:1.

In embodiments wherein the pharmaceutical composition comprises both the at least one ER modulating drug and the at least one additional cancer therapy, they may be present in the pharmaceutical composition in a variety of molar ratios. The molar ratio between the at least one ER modulating drug and the at least one additional cancer therapy may be 1:1, or 1:15, or from 5:1 to 1:10, or from 1:1 to 1:5. The molar ratio between the at least one ER modulating drug and the at least one additional cancer therapy may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one additional cancer therapy may be less than 20:1, less than 15:1, less than 10:1, less than 9:1, less than 8:1, less than 7:1, less than 6:1, less than 5:1, less than 4:1, less than 3:1, less than 2:1, or less than 1:1.

### 7. Administration

The brain-excluded ER modulating drug as detailed herein, or the pharmaceutical compositions comprising the same, may be administered to a subject. The brain-excluded ER modulating drug with the brain-penetrating ER agonist as detailed herein, or the pharmaceutical compositions comprising the same, may be administered to a subject. The brain-excluded ER modulating drug with the brain-penetrating AR modulator as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed brain-excluded ER modulating drug, with or without a brain-penetrating ER agonist and/or a brain-penetrating AR modulator, or compositions comprising the same, may be administered to a subject by different routes including orally, ocularly, nasally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In some embodiments, administration is via aerosol or suppository. In certain embodiments, the brain-excluded ER modulating drug, with or without a brain-penetrating ER agonist and/or a brain-penetrating AR modulator, or compositions comprising the same, is administered to a subject orally, intravenously, vaginally, nasally, or transdermally, or a combination thereof. For example, administration may be orally, via injection, or transdermally via a patch. The composition may be injected into any organ or tissue of the subject. In some embodiments, the brain-excluded ER modulating drug, with or without a brain-penetrating ER agonist and/or a brain-penetrating AR modulator, or compositions comprising the same, is administered to the subject by vaginal ring administration.

In some embodiments, the ER modulating drug is administered either alone or in combination with one or more brain-penetrating ER agonists. The at least one ER modulating drug and the at least one brain-penetrating ER agonist may be administered in a variety of molar ratios. The molar ratio between the at least one ER modulating drug and the at least one brain-penetrating ER agonist may be 1:1, or 1:15, or from 5:1 to 1:10, or from 1:1 to 1:5. The molar ratio between the at least one ER modulating drug and the at least one brain-penetrating ER agonist may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one brain-penetrating ER agonist may be less than 20:1, less than 15:1, less than 10:1, less than 9:1, less than 8:1, less than 7:1, less than 6:1, less than 5:1, less than 4:1, less than 3:1, less than 2:1, or less than 1:1.

In some embodiments, the ER modulating drug is administered either alone or in combination with one or more brain-penetrating AR modulators. The at least one ER modulating drug and the at least one brain-penetrating AR modulator may be administered in a variety of molar ratios. The molar ratio between the at least one ER modulating drug and the at least one brain-penetrating AR modulator may be 1:1, or 1:15, or from 5:1 to 1:10, or from 1:1 to 1:5. The molar ratio between the at least one ER modulating drug and the at least one brain-penetrating AR modulator may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one brain-penetrating AR modulator may be less than 20:1, less than 15:1, less than 10:1, less than 9:1, less than 8:1, less than 7:1, less than 6:1, less than 5:1, less than 4:1, less than 3:1, less than 2:1, or less than 1:1.

In some embodiments, the ER modulating drug is administered either alone or in combination with one or more additional therapies. The at least one ER modulating drug and the at least one additional cancer therapy may be administered in a variety of molar ratios. The molar ratio between the at least one ER modulating drug and the at least one additional cancer therapy may be 1:1, or 1:15, or from 5:1 to 1:10, or from 1:1 to 1:5. The molar ratio between the at least one ER modulating drug and the at least one additional cancer therapy may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the at least one ER modulating drug and the at least one additional cancer therapy may be less than 20:1, less than 15:1, less than 10:1, less than 9:1, less than 8:1, less than 7:1, less than 6:1, less than 5:1, less than 4:1, less than 3:1, less than 2:1, or less than 1:1.

In some embodiments, the ER modulating drug is administered to the subject by oral administration (orally, or “os”). In some embodiments, the ER modulating drug is administered to the subject in a dose every day (daily). In some embodiments, the brain-penetrating ER agonist and/or brain-penetrating AR modulator is administered to the subject by oral administration (orally, or “os”). In some embodiments, the brain-penetrating ER agonist and/or brain-penetrating AR modulator is administered to the subject in a dose every day (daily).

In certain embodiments, ER modulating drug is administered at about 0.5 mg/day per os to about 1 g/day per os, or at about 0.5 mg/day per os to about 10 mg/day per os, such as about 0.5 mg/day per os to about 5 mg/day per os, about 0.5 mg/day per os to about 5 mg/day per os, about 1 mg/day per os to about 5 mg/day per os, about 2 mg/day per os to about 5 mg/day per os, about 3 mg/day per os to about 5 mg/day per os, about 4 mg/day per os to about 5 mg/day per os, about 0.5 mg/day per os to about 4 mg/day per os, about 1 mg/day per os to about 4 mg/day per os, about 2 mg/day per os to about 4 mg/day per os, about 3 mg/day per os to about 4 mg/day per os, about 0.5 mg/day per os to about 3 mg/day per os, about 1 mg/day per os to about 3 mg/day per os, about 2 mg/day per os to about 3 mg/day per os, about 0.5 mg/day per os to about 2 mg/day per os, about 1 mg/day per os to about 2 mg/day per os, or about 0.5 mg/day per os to about 1 mg/day per os. In certain embodiments, ER modulating drug is administered at less than about 0.2 mg/day, less than about 0.25 mg/day, less than about 0.3 mg/day, less than about 0.35 mg/day, less than about 0.4 mg/day, less than about 0.45 mg/day, less than about 0.5 mg/day, less than about 0.55 mg/day, less than about 0.6 mg/day, less than about 0.65 mg/day, less than about 0.7 mg/day, less than about 0.75 mg/day, less than about 0.8 mg/day, less than about 0.85 mg/day, less than about 0.9 mg/day, less than about 0.95 mg/day, less than about 1.0 mg/day, less than about 1.5 mg/day, less than about 2.0 mg/day, less than about 2.5 mg/day, less than about 3.0 mg/day, less than about 3.5 mg/day, less than about 4.0 mg/day, less than about 4.5 mg/day, less than about 5.0 mg/day, less than about 5.5 mg/day, less than about 6.0 mg/day, less than about 6.5 mg/day, less than about 7.0 mg/day, less than about 7.5 mg/day, less than about 8.0 mg/day, less than about 8.5 mg/day, less than about 9.0 mg/day, less than about 9.5 mg/day, or less than about 10.0 mg/day. In certain embodiments, ER modulating drug is administered at at least about 0.2 mg/day, at least about 0.25 mg/day, at least about 0.3 mg/day, at least about 0.35 mg/day, at least about 0.4 mg/day, at least about 0.45 mg/day, at least about 0.5 mg/day, at least about 0.55 mg/day, at least about 0.6 mg/day, at least about 0.65 mg/day, at least about 0.7 mg/day, at least about 0.75 mg/day, at least about 0.8 mg/day, at least about 0.85 mg/day, at least about 0.9 mg/day, at least about 0.95 mg/day, or at least about 1.0 mg/day. In some embodiments, the ER modulating drug is administered at about 0.5 mg/day per os. In some embodiments, the ER modulating drug is administered at about 1 mg/day per os. In some embodiments, the ER modulating drug is administered at about 1.5 mg/day per os. In some embodiments, the ER modulating drug is administered at about 2 mg/day per os. In some embodiments, the ER modulating drug is administered at about 2.5 mg/day per os. In some embodiments, the ER modulating drug is administered at about 3 mg/day per os. In some embodiments, the ER modulating drug is administered at about 3.5 mg/day per os. In some embodiments, the ER modulating drug is administered at about 4 mg/day per os. In some embodiments, the ER modulating drug is administered at about 4.5 mg/day per os. In some embodiments, the ER modulating drug is administered at about 5 mg/day per os. In some embodiments, the ER modulating drug is administered at about 6 mg/day per os. In some embodiments, the ER modulating drug is administered at about 7 mg/day per os. In some embodiments, the ER modulating drug is administered at about 8 mg/day per os. In some embodiments, the ER modulating drug is administered at about 9 mg/day per os. In some embodiments, the ER modulating drug is administered at about 10 mg/day per os. In some other embodiments, the ER modulating drug is administered at more than 10 mg/day per os.

The ER modulating drug may be administered to a subject in a dose of about 0.5 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg.

The ER modulating drug may be administered to a subject in a dose of less than about 0.5 mg, less than about 1.0 mg, less than about 1.5 mg, less than about 2.0 mg, less than about 2.5 mg, less than about 3.0 mg, less than about 3.5 mg, less than about 4.0 mg, less than about 4.5 mg, less than about 5.0 mg, less than about 5.5 mg, less than about 6.0 mg, less than about 6.5 mg, less than about 7.0 mg, less than about 7.5 mg, less than about 8.0 mg, less than about 8.5 mg, less than about 9.0 mg, less than about 9.5 mg, less than about 10 mg, less than about 11 mg, less than about 12 mg, less than about 13 mg, less than about 14 mg, less than about 15 mg, less than about 16 mg, less than about 17 mg, less than about 18 mg, less than about 19 mg, less than about 20 mg, less than about 25 mg, less than about 30 mg, less than about 35 mg, less than about 40 mg, less than about 45 mg, less than about 50 mg, less than about 55 mg, less than about 60 mg, less than about 65 mg, less than about 70 mg, less than about 75 mg, less than about 80 mg, less than about 85 mg, less than about 90 mg, less than about 95 mg, less than about 100 mg, less than about 105 mg, less than about 110 mg, less than about 115 mg, less than about 120 mg, less than about 125 mg, less than about 130 mg, less than about 135 mg, less than about 140 mg, less than about 145 mg, less than about 150 mg, less than about 155 mg, less than about 160 mg, less than about 165 mg, less than about 170 mg, less than about 175 mg, less than about 180 mg, less than about 185 mg, less than about 190 mg, less than about 195 mg, less than about 200 mg, less than about 205 mg, less than about 210 mg, less than about 215 mg, less than about 220 mg, less than about 225 mg, less than about 230 mg, less than about 235 mg, less than about 240 mg, less than about 245 mg, less than about 250 mg, less than about 255 mg, less than about 260 mg, less than about 265 mg, less than about 270 mg, less than about 275 mg, less than about 280 mg, less than about 285 mg, less than about 290 mg, less than about 295 mg, less than about 300 mg, less than about 305 mg, less than about 310 mg, less than about 315 mg, less than about 320 mg, less than about 325 mg, less than about 330 mg, less than about 335 mg, less than about 340 mg, less than about 345 mg, less than about 350 mg, less than about 355 mg, less than about 360 mg, less than about 365 mg, less than about 370 mg, less than about 375 mg, less than about 380 mg, less than about 385 mg, less than about 390 mg, less than about 395 mg, less than about 400 mg, less than about 405 mg, less than about 410 mg, less than about 415 mg, less than about 420 mg, less than about 425 mg, less than about 430 mg, less than about 435 mg, less than about 440 mg, less than about 445 mg, less than about 450 mg, less than about 455 mg, less than about 460 mg, less than about 465 mg, less than about 470 mg, less than about 475 mg, less than about 480 mg, less than about 485 mg, less than about 490 mg, less than about 495 mg, less than about 500 mg, less than about 505 mg, less than about 510 mg, less than about 515 mg, less than about 520 mg, less than about 525 mg, less than about 530 mg, less than about 535 mg, less than about 540 mg, less than about 545 mg, less than about 550 mg, less than about 555 mg, less than about 560 mg, less than about 565 mg, less than about 570 mg, less than about 575 mg, less than about 580 mg, less than about 585 mg, less than about 590 mg, less than about 595 mg, less than about 600 mg, less than about 605 mg, less than about 610 mg, less than about 615 mg, less than about 620 mg, less than about 625 mg, less than about 630 mg, less than about 635 mg, less than about 640 mg, less than about 645 mg, less than about 650 mg, less than about 655 mg, less than about 660 mg, less than about 665 mg, less than about 670 mg, less than about 675 mg, less than about 680 mg, less than about 685 mg, less than about 690 mg, less than about 695 mg, less than about 700 mg, less than about 705 mg, less than about 710 mg, less than about 715 mg, less than about 720 mg, less than about 725 mg, less than about 730 mg, less than about 735 mg, less than about 740 mg, less than about 745 mg, less than about 750 mg, less than about 755 mg, less than about 760 mg, less than about 765 mg, less than about 770 mg, less than about 775 mg, less than about 780 mg, less than about 785 mg, less than about 790 mg, less than about 795 mg, less than about 800 mg, less than about 805 mg, less than about 810 mg, less than about 815 mg, less than about 820 mg, less than about 825 mg, less than about 830 mg, less than about 835 mg, less than about 840 mg, less than about 845 mg, less than about 850 mg, less than about 855 mg, less than about 860 mg, less than about 865 mg, less than about 870 mg, less than about 875 mg, less than about 880 mg, less than about 885 mg, less than about 890 mg, less than about 895 mg, less than about 900 mg, less than about 905 mg, less than about 910 mg, less than about 915 mg, less than about 920 mg, less than about 925 mg, less than about 930 mg, less than about 935 mg, less than about 940 mg, less than about 945 mg, less than about 950 mg, less than about 955 mg, less than about 960 mg, less than about 965 mg, less than about 970 mg, less than about 975 mg, less than about 980 mg, less than about 985 mg, less than about 990 mg, less than about 995 mg, or less than about 1000 mg. [000100] The ER modulating drug may be administered to a subject in a dose of at least about 0.5 mg, at least about 1.0 mg, at least about 1.5 mg, at least about 2.0 mg, at least about 2.5 mg, at least about 3.0 mg, at least about 3.5 mg, at least about 4.0 mg, at least about 4.5 mg, at least about 5.0 mg, at least about 5.5 mg, at least about 6.0 mg, at least about 6.5 mg, at least about 7.0 mg, at least about 7.5 mg, at least about 8.0 mg, at least about 8.5 mg, at least about 9.0 mg, at least about 9.5 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, at least about 205 mg, at least about 210 mg, at least about 215 mg, at least about 220 mg, at least about 225 mg, at least about 230 mg, at least about 235 mg, at least about 240 mg, at least about 245 mg, at least about 250 mg, at least about 255 mg, at least about 260 mg, at least about 265 mg, at least about 270 mg, at least about 275 mg, at least about 280 mg, at least about 285 mg, at least about 290 mg, at least about 295 mg, at least about 300 mg, at least about 305 mg, at least about 310 mg, at least about 315 mg, at least about 320 mg, at least about 325 mg, at least about 330 mg, at least about 335 mg, at least about 340 mg, at least about 345 mg, at least about 350 mg, at least about 355 mg, at least about 360 mg, at least about 365 mg, at least about 370 mg, at least about 375 mg, at least about 380 mg, at least about 385 mg, at least about 390 mg, at least about 395 mg, at least about 400 mg, at least about 405 mg, at least about 410 mg, at least about 415 mg, at least about 420 mg, at least about 425 mg, at least about 430 mg, at least about 435 mg, at least about 440 mg, at least about 445 mg, at least about 450 mg, at least about 455 mg, at least about 460 mg, at least about 465 mg, at least about 470 mg, at least about 475 mg, at least about 480 mg, at least about 485 mg, at least about 490 mg, at least about 495 mg, at least about 500 mg, at least about 505 mg, at least about 510 mg, at least about 515 mg, at least about 520 mg, at least about 525 mg, at least about 530 mg, at least about 535 mg, at least about 540 mg, at least about 545 mg, at least about 550 mg, at least about 555 mg, at least about 560 mg, at least about 565 mg, at least about 570 mg, at least about 575 mg, at least about 580 mg, at least about 585 mg, at least about 590 mg, at least about 595 mg, at least about 600 mg, at least about 605 mg, at least about 610 mg, at least about 615 mg, at least about 620 mg, at least about 625 mg, at least about 630 mg, at least about 635 mg, at least about 640 mg, at least about 645 mg, at least about 650 mg, at least about 655 mg, at least about 660 mg, at least about 665 mg, at least about 670 mg, at least about 675 mg, at least about 680 mg, at least about 685 mg, at least about 690 mg, at least about 695 mg, at least about 700 mg, at least about 705 mg, at least about 710 mg, at least about 715 mg, at least about 720 mg, at least about 725 mg, at least about 730 mg, at least about 735 mg, at least about 740 mg, at least about 745 mg, at least about 750 mg, at least about 755 mg, at least about 760 mg, at least about 765 mg, at least about 770 mg, at least about 775 mg, at least about 780 mg, at least about 785 mg, at least about 790 mg, at least about 795 mg, at least about 800 mg, at least about 805 mg, at least about 810 mg, at least about 815 mg, at least about 820 mg, at least about 825 mg, at least about 830 mg, at least about 835 mg, at least about 840 mg, at least about 845 mg, at least about 850 mg, at least about 855 mg, at least about 860 mg, at least about 865 mg, at least about 870 mg, at least about 875 mg, at least about 880 mg, at least about 885 mg, at least about 890 mg, at least about 895 mg, at least about 900 mg, at least about 905 mg, at least about 910 mg, at least about 915 mg, at least about 920 mg, at least about 925 mg, at least about 930 mg, at least about 935 mg, at least about 940 mg, at least about 945 mg, at least about 950 mg, at least about 955 mg, at least about 960 mg, at least about 965 mg, at least about 970 mg, at least about 975 mg, at least about 980 mg, at least about 985 mg, at least about 990 mg, at least about 995 mg, or at least about 1000 mg.

The ER modulating drug may be administered to a subject in a dose of about 0.5 mg to about 1000 mg, about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 10 mg to about 200 mg, about 10 mg to about 100 mg, or about 10 mg to about 50 mg.

In certain embodiments, when the ER modulating drug is administered to a subject whose cancer has not acquired endocrine resistance, the ER modulating drug can be administered at less than 0.5 mg/day per os for prevention of endocrine resistance. In certain embodiments, when the ER modulating drug is administered to cancer patient as adjuvant treatment, the ER modulating drug can be administered at less than 0.5 mg/day per os for prevention of endocrine resistance.

The brain-penetrating ER agonist or brain-penetrating AR modulator may be administered to a subject in a dose of about 1.0 μg, about 1.5 μg, about 2.0 μg, about 2.5 μg, about 3.0 μg, about 3.5 μg, about 4.0 μg, about 4.5 μg, about 5.0 μg, about 5.5 μg, about 6.0 μg, about 6.5 μg, about 7.0 μg, about 7.5 μg, about 8.0 μg, about 8.5 μg, about 9.0 μg, about 9.5 μg, about 10 μg, about 11 μg, about 12 μg, about 13 μg, about 14 μg, about 15 μg, about 16 μg, about 17 μg, about 18 μg, about 19 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, about 500 μg, about 505 μg, about 510 μg, about 515 μg, about 520 μg, about 525 μg, about 530 μg, about 535 μg, about 540 μg, about 545 μg, about 550 μg, about 555 μg, about 560 μg, about 565 μg, about 570 μg, about 575 μg, about 580 μg, about 585 μg, about 590 μg, about 595 μg, about 600 μg, about 605 μg, about 610 μg, about 615 μg, about 620 μg, about 625 μg, about 630 μg, about 635 μg, about 640 μg, about 645 μg, about 650 μg, about 655 μg, about 660 μg, about 665 μg, about 670 μg, about 675 μg, about 680 μg, about 685 μg, about 690 μg, about 695 μg, about 700 μg, about 705 μg, about 710 μg, about 715 μg, about 720 μg, about 725 μg, about 730 μg, about 735 μg, about 740 μg, about 745 μg, about 750 μg, about 755 μg, about 760 μg, about 765 μg, about 770 μg, about 775 μg, about 780 μg, about 785 μg, about 790 μg, about 795 μg, about 800 μg, about 805 μg, about 810 μg, about 815 μg, about 820 μg, about 825 μg, about 830 μg, about 835 μg, about 840 μg, about 845 μg, about 850 μg, about 855 μg, about 860 μg, about 865 μg, about 870 μg, about 875 μg, about 880 μg, about 885 μg, about 890 μg, about 895 μg, about 900 μg, about 905 μg, about 910 μg, about 915 μg, about 920 μg, about 925 μg, about 930 μg, about 935 μg, about 940 μg, about 945 μg, about 950 μg, about 955 μg, about 960 μg, about 965 μg, about 970 μg, about 975 μg, about 980 μg, about 985 μg, about 990 μg, about 995 μg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, about 6.0 mg, about 6,5 mg, about 7.0 mg, about 7,5 mg, about 8.0 mg, about 8.5 mg, about 9.0 mg, about 9.5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg. [000104] The brain-penetrating ER agonist or brain-penetrating AR modulator may be administered to a subject in a dose of less than about 1.0 μg, less than about 1.5 μg, less than about 2.0 μg, less than about 2.5 μg, less than about 3.0 μg, less than about 3.5 μg, less than about 4.0 μg, less than about 4.5 μg, less than about 5.0 μg, less than about 5.5 μg, less than about 6.0 μg, less than about 6.5 μg, less than about 7.0 μg, less than about 7.5 μg, less than about 8.0 μg, less than about 8.5 μg, less than about 9.0 μg, less than about 9.5 μg, less than about 10 μg, less than about 11 μg, less than about 12 μg, less than about 13 μg, less than about 14 μg, less than about 15 μg, less than about 16 μg, less than about 17 μg, less than about 18 μg, less than about 19 μg, less than about 20 μg, less than about 25 μg, less than about 30 μg, less than about 35 μg, less than about 40 μg, less than about 45 μg, less than about 50 μg, less than about 55 μg, less than about 60 μg, less than about 65 μg, less than about 70 μg, less than about 75 μg, less than about 80 μg, less than about 85 μg, less than about 90 μg, less than about 95 μg, less than about 100 μg, less than about 105 μg, less than about 110 μg, less than about 115 μg, less than about 120 μg, less than about 125 μg, less than about 130 μg, less than about 135 μg, less than about 140 μg, less than about 145 μg, less than about 150 μg, less than about 155 μg, less than about 160 μg, less than about 165 μg, less than about 170 μg, less than about 175 μg, less than about 180 μg, less than about 185 μg, less than about 190 μg, less than about 195 μg, less than about 200 μg, less than about 205 μg, less than about 210 μg, less than about 215 μg, less than about 220 μg, less than about 225 μg, less than about 230 μg, less than about 235 μg, less than about 240 μg, less than about 245 μg, less than about 250 μg, less than about 255 μg, less than about 260 μg, less than about 265 μg, less than about 270 μg, less than about 275 μg, less than about 280 μg, less than about 285 μg, less than about 290 μg, less than about 295 μg, less than about 300 μg, less than about 305 μg, less than about 310 μg, less than about 315 μg, less than about 320 μg, less than about 325 μg, less than about 330 μg, less than about 335 μg, less than about 340 μg, less than about 345 μg, less than about 350 μg, less than about 355 μg, less than about 360 μg, less than about 365 μg, less than about 370 μg, less than about 375 μg, less than about 380 μg, less than about 385 μg, less than about 390 μg, less than about 395 μg, less than about 400 μg, less than about 405 μg, less than about 410 μg, less than about 415 μg, less than about 420 μg, less than about 425 μg, less than about 430 μg, less than about 435 μg, less than about 440 μg, less than about 445 μg, less than about 450 μg, less than about 455 μg, less than about 460 μg, less than about 465 μg, less than about 470 μg, less than about 475 μg, less than about 480 μg, less than about 485 μg, less than about 490 μg, less than about 495 μg, less than about 500 μg, less than about 505 μg, less than about 510 μg, less than about 515 μg, less than about 520 μg, less than about 525 μg, less than about 530 μg, less than about 535 μg, less than about 540 μg, less than about 545 μg, less than about 550 μg, less than about 555 μg, less than about 560 μg, less than about 565 μg, less than about 570 μg, less than about 575 μg, less than about 580 μg, less than about 585 μg, less than about 590 μg, less than about 595 μg, less than about 600 μg, less than about 605 μg, less than about 610 μg, less than about 615 μg, less than about 620 μg, less than about 625 μg, less than about 630 μg, less than about 635 μg, less than about 640 μg, less than about 645 μg, less than about 650 μg, less than about 655 μg, less than about 660 μg, less than about 665 μg, less than about 670 μg, less than about 675 μg, less than about 680 μg, less than about 685 μg, less than about 690 μg, less than about 695 μg, less than about 700 μg, less than about 705 μg, less than about 710 μg, less than about 715 μg, less than about 720 μg, less than about 725 μg, less than about 730 μg, less than about 735 μg, less than about 740 μg, less than about 745 μg, less than about 750 μg, less than about 755 μg, less than about 760 μg, less than about 765 μg, less than about 770 μg, less than about 775 μg, less than about 780 μg, less than about 785 μg, less than about 790 μg, less than about 795 μg, less than about 800 μg, less than about 805 μg, less than about 810 μg, less than about 815 μg, less than about 820 μg, less than about 825 μg, less than about 830 μg, less than about 835 μg, less than about 840 μg, less than about 845 μg, less than about 850 μg, less than about 855 μg, less than about 860 μg, less than about 865 μg, less than about 870 μg, less than about 875 μg, less than about 880 μg, less than about 885 μg, less than about 890 μg, less than about 895 μg, less than about 900 μg, less than about 905 μg, less than about 910 μg, less than about 915 μg, less than about 920 μg, less than about 925 μg, less than about 930 μg, less than about 935 μg, less than about 940 μg, less than about 945 μg, less than about 950 μg, less than about 955 μg, less than about 960 μg, less than about 965 μg, less than about 970 μg, less than about 975 μg, less than about 980 μg, less than about 985 μg, less than about 990 μg, less than about 995 μg, less than about 1.0 mg, less than about 1.5 mg, less than about 2.0 mg, less than about 2.5 mg, less than about 3.0 mg, less than about 3.5 mg, less than about 4.0 mg, less than about 4.5 mg, less than about 5.0 mg, less than about 5.5 mg, less than about 6.0 mg, less than about 6.5 mg, less than about 7.0 mg, less than about 7.5 mg, less than about 8.0 mg, less than about 8.5 mg, less than about 9.0 mg, less than about 9.5 mg, less than about 10 mg, less than about 11 mg, less than about 12 mg, less than about 13 mg, less than about 14 mg, less than about 15 mg, less than about 16 mg, less than about 17 mg, less than about 18 mg, less than about 19 mg, less than about 20 mg, less than about 25 mg, less than about 30 mg, less than about 35 mg, less than about 40 mg, less than about 45 mg, less than about 50 mg, less than about 55 mg, less than about 60 mg, less than about 65 mg, less than about 70 mg, less than about 75 mg, less than about 80 mg, less than about 85 mg, less than about 90 mg, less than about 95 mg, less than about 100 mg, less than about 105 mg, less than about 110 mg, less than about 115 mg, less than about 120 mg, less than about 125 mg, less than about 130 mg, less than about 135 mg, less than about 140 mg, less than about 145 mg, less than about 150 mg, less than about 155 mg, less than about 160 mg, less than about 165 mg, less than about 170 mg, less than about 175 mg, less than about 180 mg, less than about 185 mg, less than about 190 mg, less than about 195 mg, less than about 200 mg, less than about 205 mg, less than about 210 mg, less than about 215 mg, less than about 220 mg, less than about 225 mg, less than about 230 mg, less than about 235 mg, less than about 240 mg, less than about 245 mg, less than about 250 mg, less than about 255 mg, less than about 260 mg, less than about 265 mg, less than about 270 mg, less than about 275 mg, less than about 280 mg, less than about 285 mg, less than about 290 mg, less than about 295 mg, less than about 300 mg, less than about 305 mg, less than about 310 mg, less than about 315 mg, less than about 320 mg, less than about 325 mg, less than about 330 mg, less than about 335 mg, less than about 340 mg, less than about 345 mg, less than about 350 mg, less than about 355 mg, less than about 360 mg, less than about 365 mg, less than about 370 mg, less than about 375 mg, less than about 380 mg, less than about 385 mg, less than about 390 mg, less than about 395 mg, less than about 400 mg, less than about 405 mg, less than about 410 mg, less than about 415 mg, less than about 420 mg, less than about 425 mg, less than about 430 mg, less than about 435 mg, less than about 440 mg, less than about 445 mg, less than about 450 mg, less than about 455 mg, less than about 460 mg, less than about 465 mg, less than about 470 mg, less than about 475 mg, less than about 480 mg, less than about 485 mg, less than about 490 mg, less than about 495 mg, less than about 500 mg, less than about 505 mg, less than about 510 mg, less than about 515 mg, less than about 520 mg, less than about 525 mg, less than about 530 mg, less than about 535 mg, less than about 540 mg, less than about 545 mg, less than about 550 mg, less than about 555 mg, less than about 560 mg, less than about 565 mg, less than about 570 mg, less than about 575 mg, less than about 580 mg, less than about 585 mg, less than about 590 mg, less than about 595 mg, less than about 600 mg, less than about 605 mg, less than about 610 mg, less than about 615 mg, less than about 620 mg, less than about 625 mg, less than about 630 mg, less than about 635 mg, less than about 640 mg, less than about 645 mg, less than about 650 mg, less than about 655 mg, less than about 660 mg, less than about 665 mg, less than about 670 mg, less than about 675 mg, less than about 680 mg, less than about 685 mg, less than about 690 mg, less than about 695 mg, less than about 700 mg, less than about 705 mg, less than about 710 mg, less than about 715 mg, less than about 720 mg, less than about 725 mg, less than about 730 mg, less than about 735 mg, less than about 740 mg, less than about 745 mg, less than about 750 mg, less than about 755 mg, less than about 760 mg, less than about 765 mg, less than about 770 mg, less than about 775 mg, less than about 780 mg, less than about 785 mg, less than about 790 mg, less than about 795 mg, less than about 800 mg, less than about 805 mg, less than about 810 mg, less than about 815 mg, less than about 820 mg, less than about 825 mg, less than about 830 mg, less than about 835 mg, less than about 840 mg, less than about 845 mg, less than about 850 mg, less than about 855 mg, less than about 860 mg, less than about 865 mg, less than about 870 mg, less than about 875 mg, less than about 880 mg, less than about 885 mg, less than about 890 mg, less than about 895 mg, less than about 900 mg, less than about 905 mg, less than about 910 mg, less than about 915 mg, less than about 920 mg, less than about 925 mg, less than about 930 mg, less than about 935 mg, less than about 940 mg, less than about 945 mg, less than about 950 mg, less than about 955 mg, less than about 960 mg, less than about 965 mg, less than about 970 mg, less than about 975 mg, less than about 980 mg, less than about 985 mg, less than about 990 mg, less than about 995 mg, or less than about 1000 mg.

The brain-penetrating ER agonist or brain-penetrating AR modulator may be administered to a subject in a dose of at least about 1.0 μg, at least about 1.5 μg, at least about 2.0 μg, at least about 2.5 μg, at least about 3.0 μg, at least about 3.5 μg, at least about 4.0 μg, at least about 4.5 μg, at least about 5.0 μg, at least about 5.5 μg, at least about 6.0 μg, at least about 6.5 μg, at least about 7.0 μg, at least about 7.5 μg, at least about 8.0 μg, at least about 8.5 μg, at least about 9.0 μg, at least about 9.5 μg, at least about 10 μg, at least about 11 μg, at least about 12 μg, at least about 13 μg, at least about 14 μg, at least about 15 μg, at least about 16 μg, at least about 17 μg, at least about 18 μg, at least about 19 μg, at least about 20 μg, at least about 25 μg, at least about 30 μg, at least about 35 μg, at least about 40 μg, at least about 45 μg, at least about 50 μg, at least about 55 μg, at least about 60 μg, at least about 65 μg, at least about 70 μg, at least about 75 μg, at least about 80 μg, at least about 85 μg, at least about 90 μg, at least about 95 μg, at least about 100 μg, at least about 105 μg, at least about 110 μg, at least about 115 μg, at least about 120 μg, at least about 125 μg, at least about 130 μg, at least about 135 μg, at least about 140 μg, at least about 145 μg, at least about 150 μg, at least about 155 μg, at least about 160 μg, at least about 165 μg, at least about 170 μg, at least about 175 μg, at least about 180 μg, at least about 185 μg, at least about 190 μg, at least about 195 μg, at least about 200 μg, at least about 205 μg, at least about 210 μg, at least about 215 μg, at least about 220 μg, at least about 225 μg, at least about 230 μg, at least about 235 μg, at least about 240 μg, at least about 245 μg, at least about 250 μg, at least about 255 μg, at least about 260 μg, at least about 265 μg, at least about 270 μg, at least about 275 μg, at least about 280 μg, at least about 285 μg, at least about 290 μg, at least about 295 μg, at least about 300 μg, at least about 305 μg, at least about 310 μg, at least about 315 μg, at least about 320 μg, at least about 325 μg, at least about 330 μg, at least about 335 μg, at least about 340 μg, at least about 345 μg, at least about 350 μg, at least about 355 μg, at least about 360 μg, at least about 365 μg, at least about 370 μg, at least about 375 μg, at least about 380 μg, at least about 385 μg, at least about 390 μg, at least about 395 μg, at least about 400 μg, at least about 405 μg, at least about 410 μg, at least about 415 μg, at least about 420 μg, at least about 425 μg, at least about 430 μg, at least about 435 μg, at least about 440 μg, at least about 445 μg, at least about 450 μg, at least about 455 μg, at least about 460 μg, at least about 465 μg, at least about 470 μg, at least about 475 μg, at least about 480 μg, at least about 485 μg, at least about 490 μg, at least about 495 μg, at least about 500 μg, at least about 505 μg, at least about 510 μg, at least about 515 μg, at least about 520 μg, at least about 525 μg, at least about 530 μg, at least about 535 μg, at least about 540 μg, at least about 545 μg, at least about 550 μg, at least about 555 μg, at least about 560 μg, at least about 565 μg, at least about 570 μg, at least about 575 μg, at least about 580 μg, at least about 585 μg, at least about 590 μg, at least about 595 μg, at least about 600 μg, at least about 605 μg, at least about 610 μg, at least about 615 μg, at least about 620 μg, at least about 625 μg, at least about 630 μg, at least about 635 μg, at least about 640 μg, at least about 645 μg, at least about 650 μg, at least about 655 μg, at least about 660 μg, at least about 665 μg, at least about 670 μg, at least about 675 μg, at least about 680 μg, at least about 685 μg, at least about 690 μg, at least about 695 μg, at least about 700 μg, at least about 705 μg, at least about 710 μg, at least about 715 μg, at least about 720 μg, at least about 725 μg, at least about 730 μg, at least about 735 μg, at least about 740 μg, at least about 745 μg, at least about 750 μg, at least about 755 μg, at least about 760 μg, at least about 765 μg, at least about 770 μg, at least about 775 μg, at least about 780 μg, at least about 785 μg, at least about 790 μg, at least about 795 μg, at least about 800 μg, at least about 805 μg, at least about 810 μg, at least about 815 μg, at least about 820 μg, at least about 825 μg, at least about 830 μg, at least about 835 μg, at least about 840 μg, at least about 845 μg, at least about 850 μg, at least about 855 μg, at least about 860 μg, at least about 865 μg, at least about 870 μg, at least about 875 μg, at least about 880 μg, at least about 885 μg, at least about 890 μg, at least about 895 μg, at least about 900 μg, at least about 905 μg, at least about 910 μg, at least about 915 μg, at least about 920 μg, at least about 925 μg, at least about 930 μg, at least about 935 μg, at least about 940 μg, at least about 945 μg, at least about 950 μg, at least about 955 μg, at least about 960 μg, at least about 965 μg, at least about 970 μg, at least about 975 μg, at least about 980 μg, at least about 985 μg, at least about 990 μg, at least about 995 μg, at least about 1.0 mg, at least about 1.5 mg, at least about 2.0 mg, at least about 2.5 mg, at least about 3.0 mg, at least about 3.5 mg, at least about 4.0 mg, at least about 4.5 mg, at least about 5.0 mg, at least about 5.5 mg, at least about 6.0 mg, at least about 6.5 mg, at least about 7.0 mg, at least about 7.5 mg, at least about 8.0 mg, at least about 8.5 mg, at least about 9.0 mg, at least about 9.5 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, at least about 205 mg, at least about 210 mg, at least about 215 mg, at least about 220 mg, at least about 225 mg, at least about 230 mg, at least about 235 mg, at least about 240 mg, at least about 245 mg, at least about 250 mg, at least about 255 mg, at least about 260 mg, at least about 265 mg, at least about 270 mg, at least about 275 mg, at least about 280 mg, at least about 285 mg, at least about 290 mg, at least about 295 mg, at least about 300 mg, at least about 305 mg, at least about 310 mg, at least about 315 mg, at least about 320 mg, at least about 325 mg, at least about 330 mg, at least about 335 mg, at least about 340 mg, at least about 345 mg, at least about 350 mg, at least about 355 mg, at least about 360 mg, at least about 365 mg, at least about 370 mg, at least about 375 mg, at least about 380 mg, at least about 385 mg, at least about 390 mg, at least about 395 mg, at least about 400 mg, at least about 405 mg, at least about 410 mg, at least about 415 mg, at least about 420 mg, at least about 425 mg, at least about 430 mg, at least about 435 mg, at least about 440 mg, at least about 445 mg, at least about 450 mg, at least about 455 mg, at least about 460 mg, at least about 465 mg, at least about 470 mg, at least about 475 mg, at least about 480 mg, at least about 485 mg, at least about 490 mg, at least about 495 mg, at least about 500 mg, at least about 505 mg, at least about 510 mg, at least about 515 mg, at least about 520 mg, at least about 525 mg, at least about 530 mg, at least about 535 mg, at least about 540 mg, at least about 545 mg, at least about 550 mg, at least about 555 mg, at least about 560 mg, at least about 565 mg, at least about 570 mg, at least about 575 mg, at least about 580 mg, at least about 585 mg, at least about 590 mg, at least about 595 mg, at least about 600 mg, at least about 605 mg, at least about 610 mg, at least about 615 mg, at least about 620 mg, at least about 625 mg, at least about 630 mg, at least about 635 mg, at least about 640 mg, at least about 645 mg, at least about 650 mg, at least about 655 mg, at least about 660 mg, at least about 665 mg, at least about 670 mg, at least about 675 mg, at least about 680 mg, at least about 685 mg, at least about 690 mg, at least about 695 mg, at least about 700 mg, at least about 705 mg, at least about 710 mg, at least about 715 mg, at least about 720 mg, at least about 725 mg, at least about 730 mg, at least about 735 mg, at least about 740 mg, at least about 745 mg, at least about 750 mg, at least about 755 mg, at least about 760 mg, at least about 765 mg, at least about 770 mg, at least about 775 mg, at least about 780 mg, at least about 785 mg, at least about 790 mg, at least about 795 mg, at least about 800 mg, at least about 805 mg, at least about 810 mg, at least about 815 mg, at least about 820 mg, at least about 825 mg, at least about 830 mg, at least about 835 mg, at least about 840 mg, at least about 845 mg, at least about 850 mg, at least about 855 mg, at least about 860 mg, at least about 865 mg, at least about 870 mg, at least about 875 mg, at least about 880 mg, at least about 885 mg, at least about 890 mg, at least about 895 mg, at least about 900 mg, at least about 905 mg, at least about 910 mg, at least about 915 mg, at least about 920 mg, at least about 925 mg, at least about 930 mg, at least about 935 mg, at least about 940 mg, at least about 945 mg, at least about 950 mg, at least about 955 mg, at least about 960 mg, at least about 965 mg, at least about 970 mg, at least about 975 mg, at least about 980 mg, at least about 985 mg, at least about 990 mg, at least about 995 mg, or at least about 1000 mg.

The brain-penetrating ER agonist or brain-penetrating AR modulator may be administered to a subject in a dose of about 1 μg to about 1000 mg, about 1 μg to about 1 mg, about 10 μg to about 1 mg, about 10 μg to about 900 μg, about 10 μg to about 800 μg, about 10 μg to about 700 μg, about 10 μg to about 600 μg, about 10 u to about 500 μg, about 10 μg to about 400 μg, about 10 μg to about 300 μg, about 10 μg to about 200 μg, about 10 μg to about 100 μg, or about 10 μg to about 50 μg, about 1 μg to about 900 mg, about 1 μg to about 800 mg, about 1 μg to about 700 mg, about 1 μg to about 600 mg, about 1 μg to about 500 mg, about 1 μg to about 400 mg, about 1 μg to about 300 mg, about 1 μg to about 200 mg, about 1 μg to about 100 mg, or about 1 μg to about 50 mg, about 1 mg to about 1000 mg, about 10 mg to about 1000 mg, about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 10 mg to about 200 mg, about 10 mg to about 100 mg, or about 10 mg to about 50 mg.

A composition can be administered alone or in combination with other treatments, either simultaneously or sequentially, dependent upon the condition to be treated. The at least one brain-excluded ER modulating drug and the at least one brain ER agonist may be administered simultaneously or sequentially. The at least one brain-excluded ER modulating drug and the at least one brain-penetrating AR modulator may be administered simultaneously or sequentially. The at least one brain-excluded ER modulating drug and the at least one additional therapy may be administered together or simultaneously, they may be administered at different times or sequentially.

The at least one brain-excluded ER modulating drug, with or without a brain-penetrating ER agonist and/or a brain-penetrating AR modulator, may be administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months. The at least one brain-excluded ER modulating drug may be administered to the subject for 1 year, 2years, 3 years, 4 years, 5 years, or more than 5 years. In some embodiments, the brain-excluded ER modulating drug, with or without a brain-penetrating ER agonist and/or a brain-penetrating AR modulator, is administered to the subject until the subject's cancer progresses on therapy.

### 8. Methods

a. Methods of Treating Cancer

Provided herein are methods of treating cancer. Provided herein are methods of treating cancer in a subject in need thereof. The methods may include administering to the subject at least one brain-excluded ER modulating drug, as detailed herein. The methods may include administering to the subject at least one brain-excluded ER modulating drug and at least one brain-penetrating ER agonist, as detailed herein. The methods may include administering to the subject at least one brain-excluded ER modulating drug and at least one brain-penetrating AR modulator, as detailed herein. The methods may further include administering to the subject at least one additional therapy, as detailed herein.

Provided herein are methods of reducing cancer metastasis. The methods may include administering to the subject at least one brain-excluded ER modulating drug, as detailed herein. The methods may include administering to the subject at least one brain-excluded ER modulating drug and at least one brain-penetrating ER agonist, as detailed herein. The methods may include administering to the subject at least one brain-excluded ER modulating drug and at least one brain-penetrating AR modulator, as detailed herein. The methods may further include administering to the subject at least one additional therapy, as detailed herein.

Provided herein are methods of reducing tumor growth. The methods may include administering to the subject at least one brain-excluded ER modulating drug, as detailed herein. The methods may include administering to the subject at least one brain-excluded ER modulating drug and at least one brain-penetrating ER agonist, as detailed herein. The methods may include administering to the subject at least one brain-excluded ER modulating drug and at least one brain-penetrating AR modulator, as detailed herein. The methods may further include administering to the subject at least one additional therapy, as detailed herein.

Provided herein are methods of reducing the size of a tumor. The methods may include administering to the subject at least one brain-excluded ER modulating drug, as detailed herein. The methods may include administering to the subject at least one brain-excluded ER modulating drug and at least one brain-penetrating ER agonist, as detailed herein. The methods may include administering to the subject at least one brain-excluded ER modulating drug and at least one brain-penetrating AR modulator, as detailed herein. The methods may further include administering to the subject at least one additional therapy, as detailed herein.

In some embodiments, the cancer is selected from breast cancer, melanoma, colon cancer, glioblastoma, ovarian cancer, head cancer, neck cancer, head and neck cancer, and lung cancer, or a combination thereof.

The compositions and methods detailed herein may have a variety of effects in the subject, relative to a control. Tumor growth may be decreased, tumor size may be decreased, the number of tumors may be reduced, circulating tumor cells may be reduced, cancer metastasis may be reduced, the number of cancer metastases may be reduced, the size of a cancer metastasis may be reduced, the growth of a cancer metastasis may be reduced, or a combination thereof.

The at least one brain-excluded ER modulating drug, with or without a brain-penetrating ER agonist and/or a brain-penetrating AR modulator, may treat cancer. The at least one brain-excluded ER modulating drug, with or without a brain-penetrating ER agonist and/or a brain-penetrating AR modulator, may reduce cancer. Reducing cancer may include reducing tumor size, reducing tumor growth, reducing the number of tumors, reducing cancer metastasis, reducing the size of a cancer metastasis, reducing the growth of a cancer metastasis, or reducing the number of cancer metastasis, or a combination thereof. In some embodiments, the brain-excluded ER modulating drug, with or without a brain-penetrating ER agonist and/or a brain-penetrating AR modulator, reduces cancer by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by about 5-95%, 10-90%, 15-85%, 20-80%, or 1.5-fold to 10-fold, relative to a control.

b. Method of Modulating an Estrogen Receptor (ER) Peripherally in a Subject but not in the Brain

Provided herein are methods of modulating an estrogen receptor (ER) peripherally in a subject but not in the brain. Modulating an ER receptor may include inhibiting an ER receptor. The methods may include administering to the subject at least one brain-excluded ER modulating drug, as detailed herein. The methods may include administering to the subject at least one brain-excluded ER modulating drug and at least one brain-penetrating ER agonist, as detailed herein. The methods may include administering to the subject at least one brain-excluded ER modulating drug and at least one brain-penetrating AR modulator, as detailed herein. The methods may further include administering to the subject at least one additional therapy, as detailed herein. The methods may include administering to the subject at least one brain-excluded estrogen receptor (ER) modulating drug such that the drug exposure in the brain is zero to minimal.

The compositions and methods detailed herein may have a variety of effects in the subject, relative to a control. Tumor growth may be decreased, tumor size may be decreased, the number of tumors may be reduced, circulating tumor cells may be reduced, cancer metastasis may be reduced, the number of cancer metastases may be reduced, the size of a cancer metastasis may be reduced, the growth of a cancer metastasis may be reduced, or a combination thereof.

The at least one brain-excluded ER modulating drug, with or without a brain-penetrating ER agonist and/or a brain-penetrating AR modulator, may treat cancer. The at least one brain-excluded ER modulating drug, with or without a brain-penetrating ER agonist and/or a brain-penetrating AR modulator, may reduce cancer. Reducing cancer may include reducing tumor size, reducing tumor growth, reducing the number of tumors, reducing cancer metastasis, reducing the size of a cancer metastasis, reducing the growth of a cancer metastasis, or reducing the number of cancer metastasis, or a combination thereof. In some embodiments, the brain-excluded ER modulating drug, with or without a brain-penetrating ER agonist and/or a brain-penetrating AR modulator, reduces cancer by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The cancer may be reduced by about 5-95%, 10-90%, 15-85%, 20-80%, or 1.5-fold to 10-fold, relative to a control. In some embodiments, the cancer is selected from breast cancer, melanoma, colon cancer, glioblastoma, ovarian cancer, head cancer, neck cancer, head and neck cancer, and lung cancer, or a combination thereof.

In some embodiments, the combination of at least one brain-excluded ER modulating drug and at least one brain-penetrating ER agonist may reduce or inhibit hot flashes in a subject. Upon administration of at least one brain-excluded ER modulating drug and at least one brain-penetrating ER agonist, the duration, and/or intensity, and/or frequency of hot flashes may be reduced. The at least one brain-excluded ER modulating drug and at least one brain-penetrating ER agonist may reduce or inhibit hot flashes by at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The at least one brain-excluded ER modulating drug and at least one brain-penetrating ER agonist may reduce or inhibit hot flashes by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The at least one brain-excluded ER modulating drug and at least one brain-penetrating ER agonist may reduce or inhibit hot flashes by about 5-95%, 10-90%, 15-85%, 20-80%, or 1.5-fold to 10-fold, relative to a control. The amelioration of hot flashes may increase patient compliance with a therapy including an ER modulating drug, relative to a therapy not including the at least one brain-penetrating ER agonist.

The ER agonist may increase the effectiveness of an ER modulating drug in treating or reducing cancer. The ER agonist may cross the blood-brain barrier, The ER agonist may activate an ER receptor in the brain.

### 9. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

All experiments were approved by the Duke University Institutional Animal Care and Use Committee (IACUC) prior to initiating work and were conducted in accordance with the Guide for the Care and Use of Animals, 8th Ed.

Prior to implantation into animals, BPD6, E0771, and A7C11 cells were cultured in DMEM media supplemented with 8% fetal bovine serum, non-essential amino acids and sodium pyruvate. Cells were trypsinized and injected in phosphate buffered saline.

For all experiments, mice were ovariectomized under anesthesia at 7 weeks of age and were provided placebo or estrogen (2.72 mcg/mL) treatment in drinking water throughout the remainder of the experiment. 7 to 10 days after surgery 0.5×10⁶ BPD6melanoma cells or E0771 (0.2×10⁶) or A7C 11 (0.4×10⁵) mammary cancer cells per mouse were injected subcutaneously (BPD6) or orthotopically (A7C11 or E0771) into the axial mammary gland. Tumor growth was monitored by caliper measurement at least 3× weekly and was calculated as L×W2×0.5. For those mice receiving treatment with SERDs, mice were randomized to treatment 2 days after tumor implantation. 3574 (camizestrant) and 3964 were administered PO QD (by mouth every day) formulated in 5% DMSO/20% hydroxypropyl-b-cyclodextrin. Fulvestrant was administered SC Q5D in 10% DMSO/95% corn oil.

At the conclusion of tumor measurement (approximately 2 weeks or when tumor reached 1.5-2 cm³ volume), mice were euthanized by CO2 exposure followed by decapitation. Uteri were removed at necropsy and weighed as a measure of estrogen stimulation.

### Example 2

### Specific Inhibition of ERα Expression in the Brain Results in E2-independent Growth of Tumors

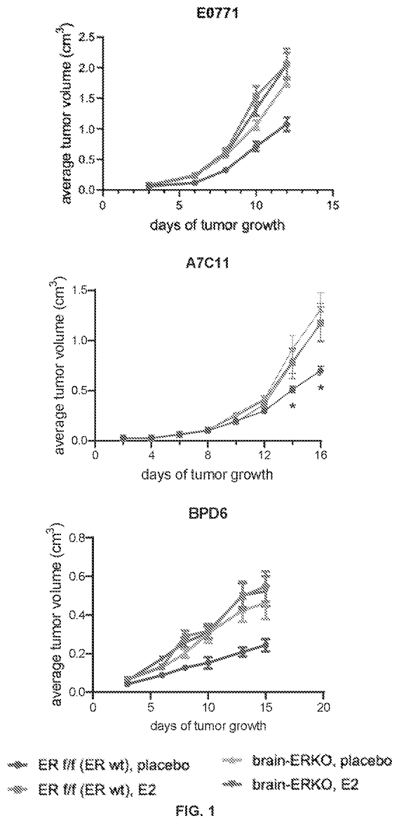

A Cre-lox genetic scheme was used to ablate ER expression in a subset of KNDY neurons in the arcuate nucleus of the basal hypothalamus (brain-ERKO). The tumor growth of 2 murine mammary cancer cell lines (E0771 and A7C 11), as well as one murine melanoma cell line (BOD6) was evaluated in ovariectomized Cre+ mice and in Cre− littermate controls receiving either placebo or estradiol (E2) treatment. Tumor volume was evaluated over 2 weeks of growth. Results are shown in FIG. 1. As shown, in the littermate (LM) controls, E2 treatment significantly increased tumor growth, whereas tumors implanted in the Cre+ mice, regardless of treatment, grew at the same rate as the estrogenized control.

Thus, loss of ER expression in the brain relieved an ER-dependent repression of tumor growth originating from the brain.

### Example 3

### Brain Penetration is an Important Distinguishing Feature of SERDs

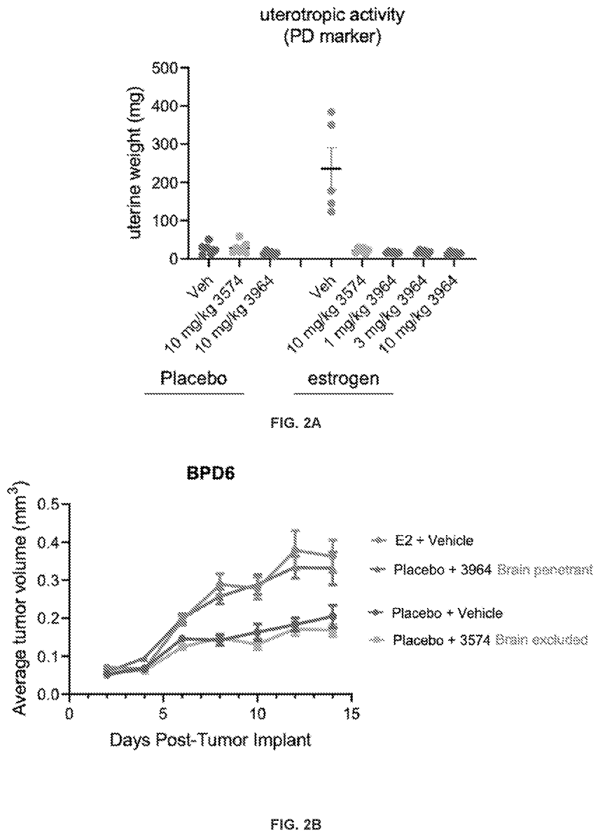

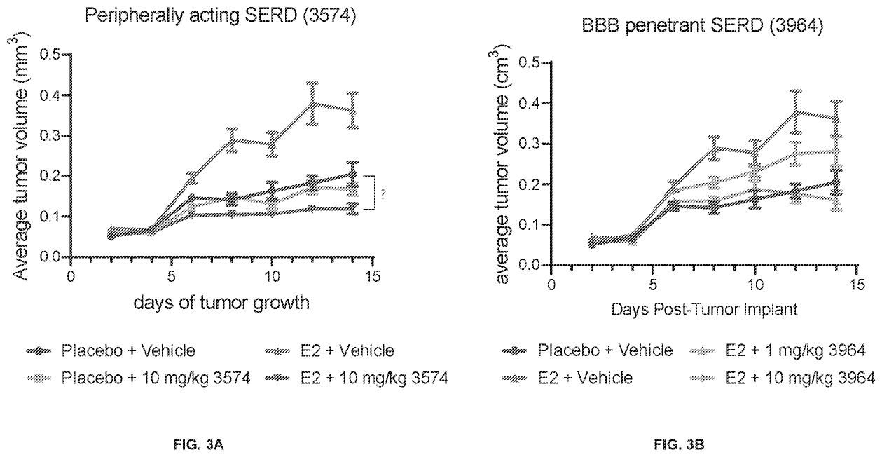

To further explore the role of brain-expressed ER in tumor growth in the periphery, 2 SERDs were used with unique pharmacokinetics. 3574 is a brain excluded SERD, affecting the peripherally expressed ER. 3964 is a brain penetrant SERD, able to inhibit/downregulate ER both peripherally and centrally. A shown on the left in FIG. 2A, neither SERD exhibited estrogenic activity using the mouse uterine weight as a sensitive measure of estrogen agonist activity, and both SERDs effectively reversed E2 stimulation of uterine weight. ER-negative BPD6 melanoma cells were implanted into ovariectomized mice receiving either placebo or estradiol (E2) treatment in drinking water. After 48 hours, mice were randomized to receive vehicle (veh) or selective estrogen receptor degrader 3574 (10 mg/kg po qd; by mouth every day). Average tumor volume +/−SEM at each day of measurement was determined. Significance (p<0.05) was determined by 2-way ANOVA followed by Tukey's multiple comparison test. Shown in FIG. 2B are the placebo treated groups and the estrogen only control, as well as both brain penetrant and excluded SERD groups without E2 treatment. The brain excluded SERD was without effect, whereas treatment with the brain penetrant SERD resulted in tumor growth similar to that observed for the E2 treated control and reminiscent of the brain-ERKO experiment on the previous SERD. That is, while estrogen treatment of BOD6 murine melanoma cells in ovariectomized C57BL6 mice receiving estradiol (“E2+Vehicle”) treatment resulted in a significant increase in tumor growth as compared to the placebo treated control (“Placebo +Vehicle”), it was found that administration of a peripheral (does not cross the blood/brain barrier) selective estrogen receptor degrader (SERD), although without significant effect in the absence of estrogen treatment (comparing “Placebo+Vehicle” and “Placebo+3574”), resulted in a significant repression of tumor growth when administered in the context of estrogen treatment. Treatment with anti-estrogens reversed this estradiol stimulation of tumor growth. E2 treated groups are included FIGS. 3A-3B.

### Example 4

### Brain Penetration is a Key Distinguishing Feature of SERDs (melanoma Model)

In comparing these SERDs (FIGS. 3A-3B), the peripheral SERD was without effect in the placebo treated animals (do no harm), but combination with E2 treatment resulted in a significant repression of tumor growth as compared to the placebo control. Although low and high doses of the brain penetrant SERD 3964 showed no difference in their peripheral reversal of E2 stimulation of the uterus, there was a clear difference in the low and high doses with respect to tumor growth. The lower dose (presumably with less brain exposure) resulted in a reversal of E2 stimulation of tumor growth, whereas increasing the SERD dose mitigated this repression so that no significant difference was noted in the E2 control and E2+10 mg/kg 3964 treatment groups. Thus, high repression/turnover of ER in the brain is unlikely to be beneficial in combating tumor growth. Finally, combination of the peripheral SERD 3574 with estradiol in fact suppressed tumor growth below that observed in the placebo/vehicle treated control. Because at this dose the SERD activity is anticipated to predominate in the periphery, these data imply that clinical benefit may be gained by engaging the ER in the brain with estrogen exposure while simultaneously suppressing ER action in the periphery with a SERD or antagonist.

### Example 5

### The Inverse Dose Response Noted is Reminiscent of that Observed in Recent Clinical Trials

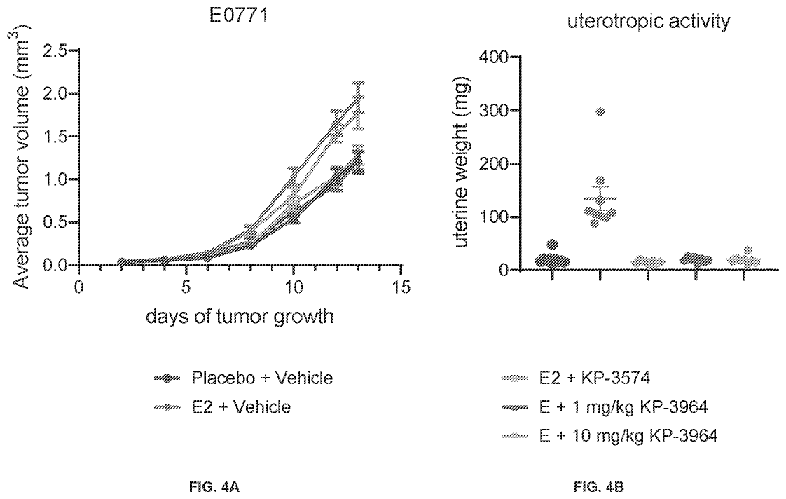

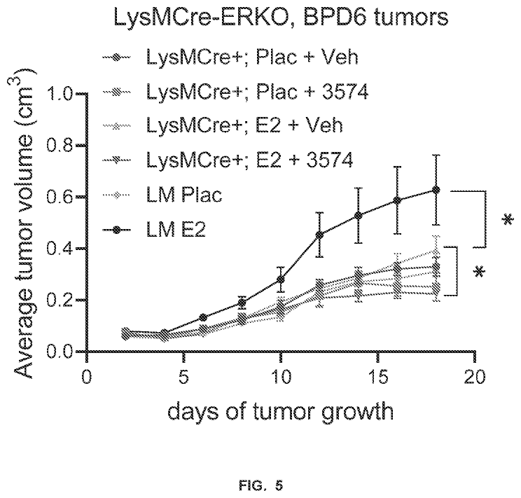

Analogous to the experiment in Example 4, E0771 mammary tumors were implanted in ovariectomized mice receiving E2 or placebo treatment. Results are shown in FIGS. 44-4B. While the brain penetrant SERD and brain excluded SERD again reversed E2 stimulation of uterine weight (right graph), as observed in the previous model, treatment with either the brain excluded SERD 3574 or with a LOW dose of the brain penetrant SERD 3964 reversed E2 stimulation of tumor growth to resemble the placebo control without E2 stimulation. Treatment with a high dose of the brain penetrant SERD resulted in growth similar to that observed in the E2 only control.

### Example 6

### Tumor Growth Is Repressed With Estrogen and a Brain-excluded SERD

It was previously determined that for some cancer subtypes, including melanoma, one mechanism by which estrogens modulate tumor growth is by promoting a pro-tumor anti-inflammatory M2-like phenotype in the tumor infiltrating macrophages. Using a genetic model employing Cre-Lox technology to specifically delete estrogen receptor in myeloid cells (including macrophages), it was previously determined that loss of estrogen receptor expression in this immune cell population results in the loss of estrogen modulation of tumor growth in these tumors. To determine whether estrogen modulation of myeloid cells contributes to the significant repression of tumor growth in E2/SERD treated tumors, a second study was performed in which these same treatments were administered to tumors bearing ERflox/flox/LysMCre+ or ERflox/flox littermate control groups. BOD6 tumors were implanted in ovariectomized ERflox/flox/LysMCre+ or ERflox/flox mice that received placebo or estradiol treatment. Mice were further randomized to receive vehicle or SERD 3574 (10 mg/kg po qd; by mouth every day). Average tumor volume +/−SEM was determined. Significance (p<0.05) was determined by 2-way ANOVA followed by Tukey's multiple comparison test. As shown in FIG. 5, while a significant increase in tumor growth was observed in estrogen treated littermate group (“LM E2”, black) as compared to the vehicle control (“LM Plac”, orange), no difference was observed in the Cre+ groups receiving estrogen (“LysMCre+; E2+Veh”, green) and placebo (“LysMCre+; Plac+Veh”, blue) treatment. While treatment with 3574 did not affect tumor growth in the absence of estrogen treatment (“LysMCre+; Plac+Veh” blue vs. “LysMCre+; Plac+3574” red), a significant repression of tumor growth in the Cre+ estrogen/3574 treatment group (“LysMCre+; E2+3574”, purple) was observed as compared to both the Cre+ placebo/vehicle (“LysMCre+; Plac+Veh”, blue) and Cre+ estrogen/vehicle control (“LysMCre+; E2+Veh”, green) groups.

### Example 7

### Fulvestrant, an Approved SERD that also Enters the Brain, also Promotes Tumor Growth in a Dose Related Manner

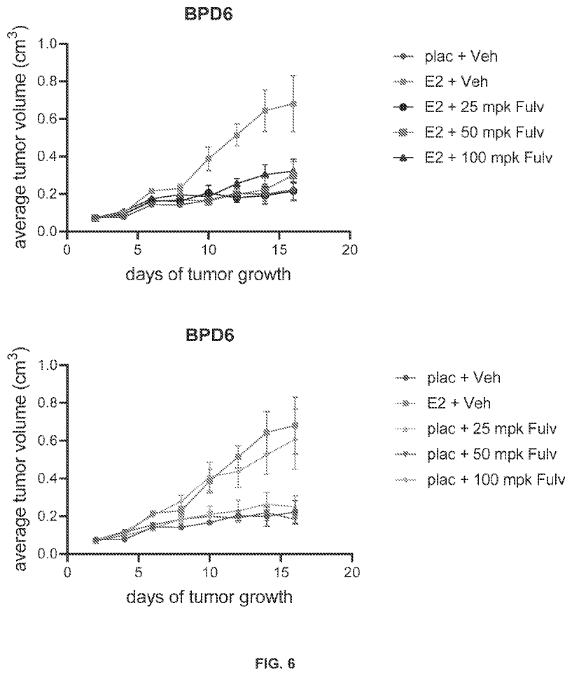

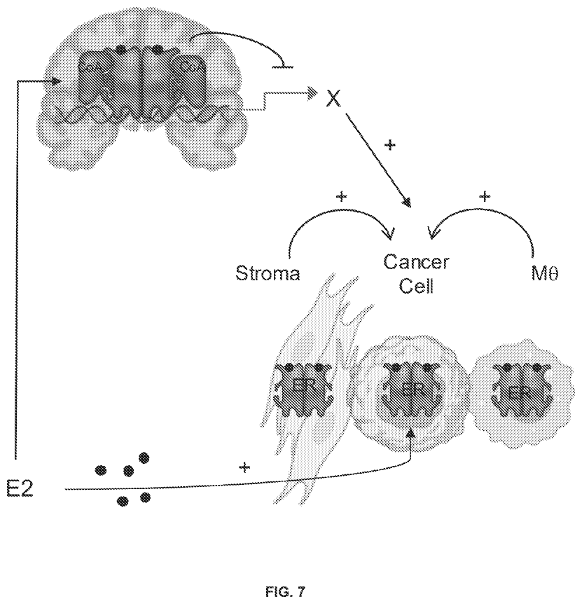

To further explore the relationship between brain exposure to SERDs and tumor response, a second melanoma experiment was conducted in which BPD6 tumors were implanted into ovariectomized mice receiving vehicle or estradiol treatment. These groups were further subdivided to receive vehicle or treatment with 25, 50, or 100 mg/kg fulvestrant. Results are shown in FIG. 6. At a high dose of fulvestrant, for which brain exposure would be highest, it was observed that tumors in mice receiving placebo+100 mg/kg fulvestrant exhibited growth similar to that of the estrogen/vehicle control. These data further imply that SERD activity in the brain is likely to circumvent tumor inhibitory pathways and enable tumor progression. A model of how peripheral and brain-penetrating SERMs/SERDs impact tumor growth is shown in FIG. 7.

### Example 8

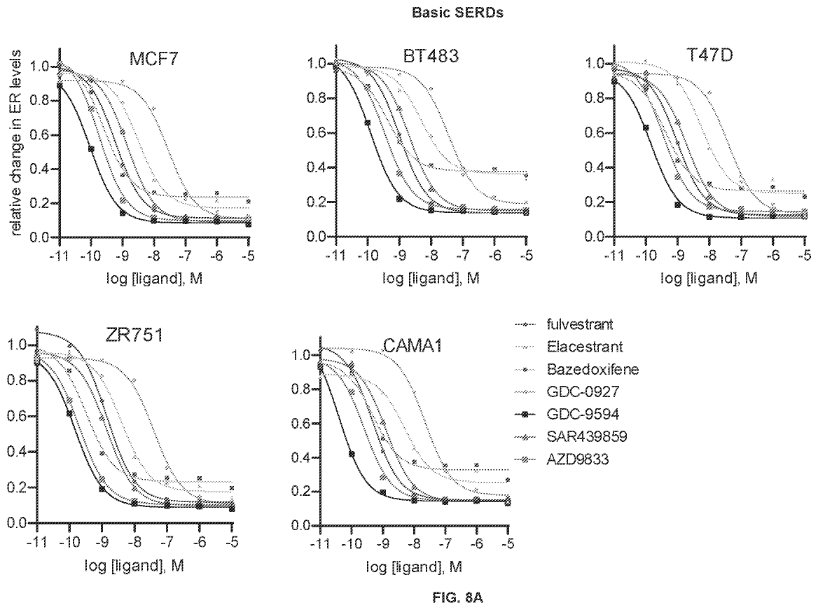

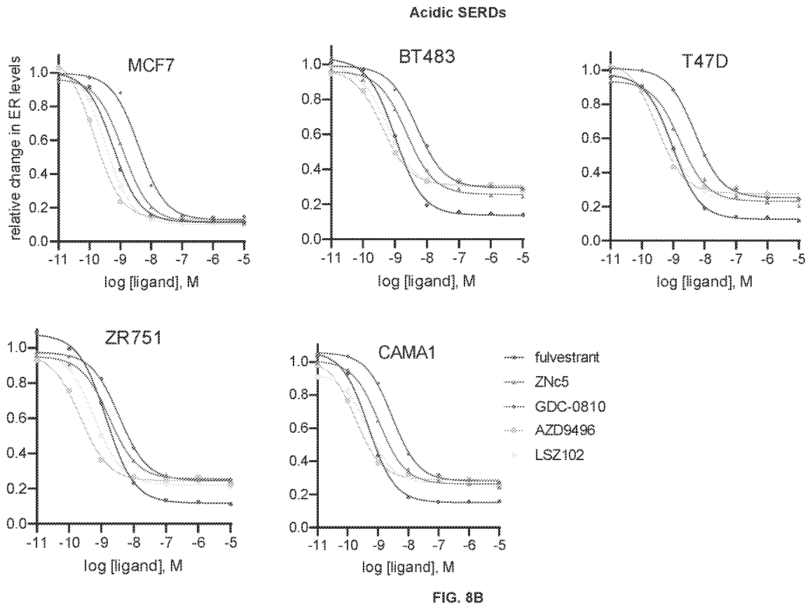

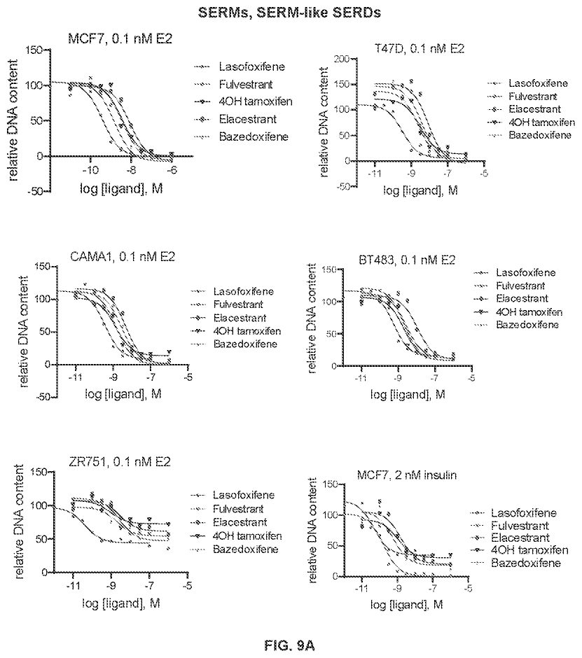

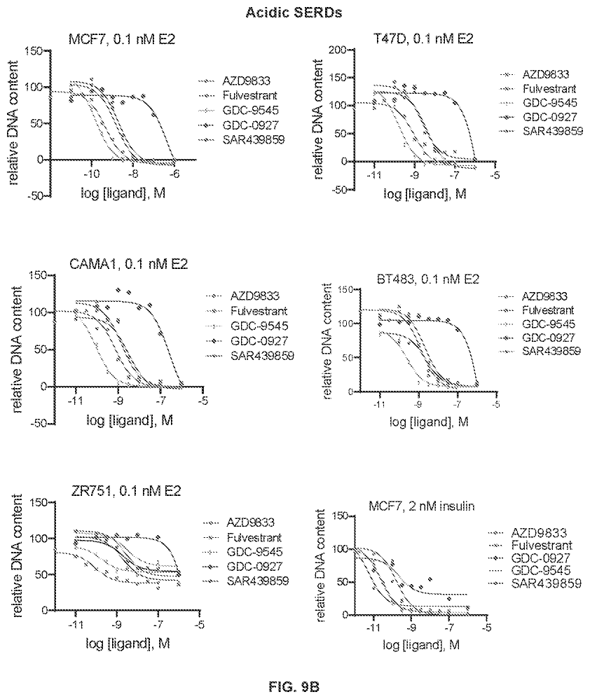

Additional experiments were completed, with results shown in FIGS. 8-13.

FIGS. 8A-8B: Using ER downregulation as an endpoint, SERDs exhibit largely similar efficacy in multiple breast cancer cells with differences noted primarily in potency. Basic SERDs degrade ER to an extent comparable to standard of care fulvestrant, while acidic SERDs show slightly less efficacy.

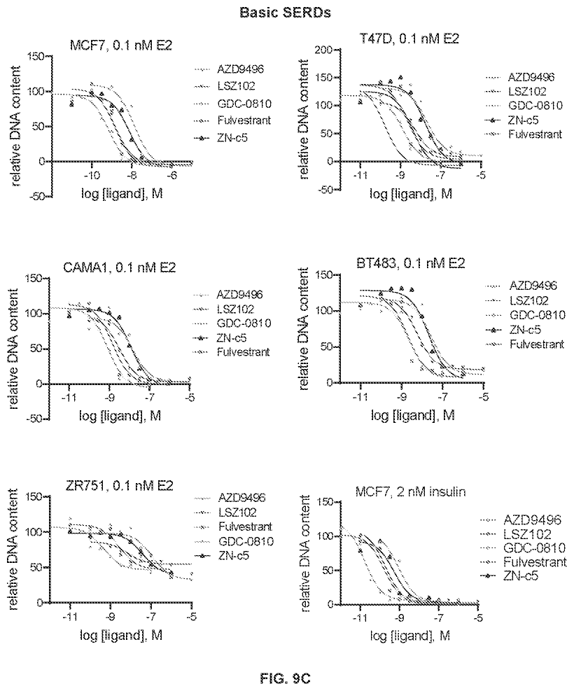

FIGS. 9A-9C: Despite differences in ER turnover noted in FIGS. 8A-8B, SERDs exhibit similar activity in breast cancer cells when using inhibition of cellular proliferation as an endpoint. Breast cancer cells show some differences in their sensitivity to ER inhibition, with MCF7 showing greatest sensitivity and ZR751 showing least sensitivity.

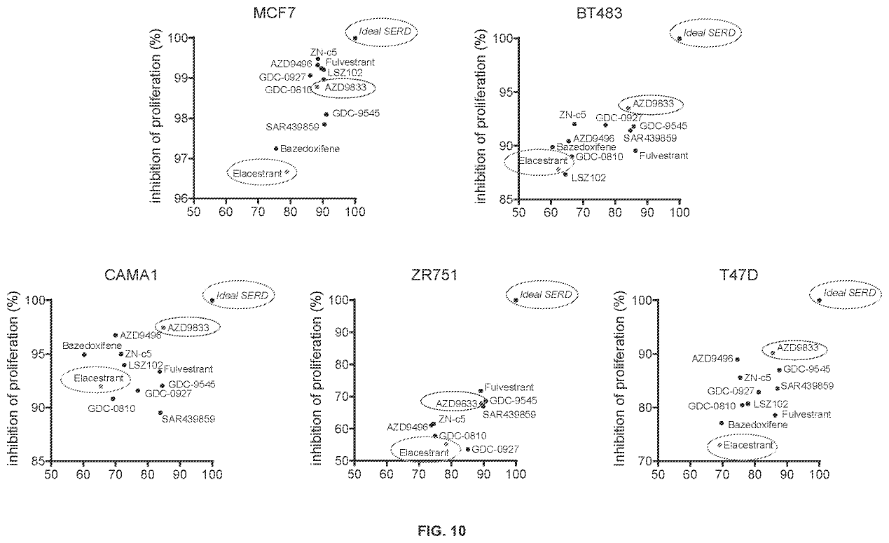

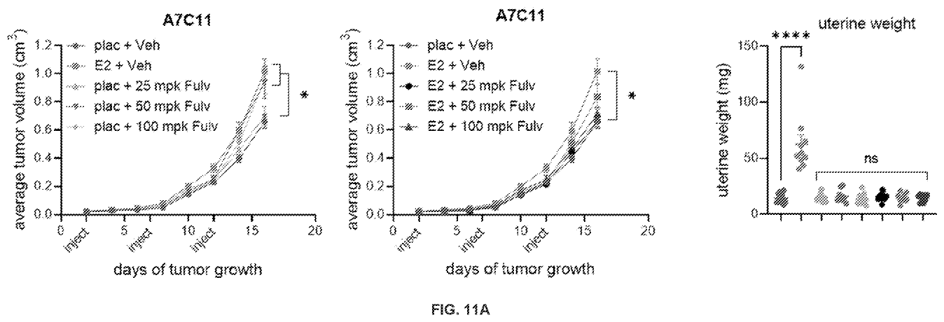

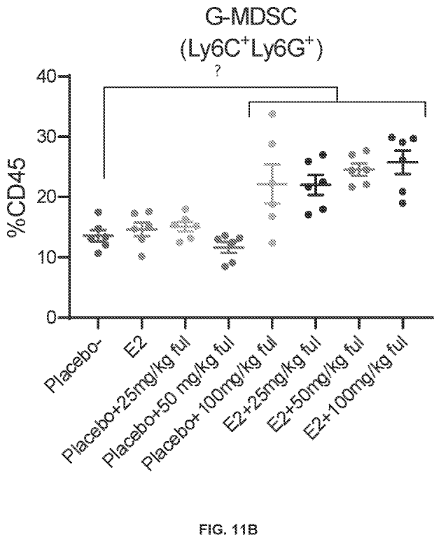

FIG. 10: ER turnover (FIGS. 8A-8B) and ER inhibition (FIGS. 9A-9C) are not proportionally related. For example, elacestrant is generally the least efficient SERD with respect to ER degradation, yet inhibits proliferation in most breast cancer cell lines efficacy similar to that observed for other SERDs.

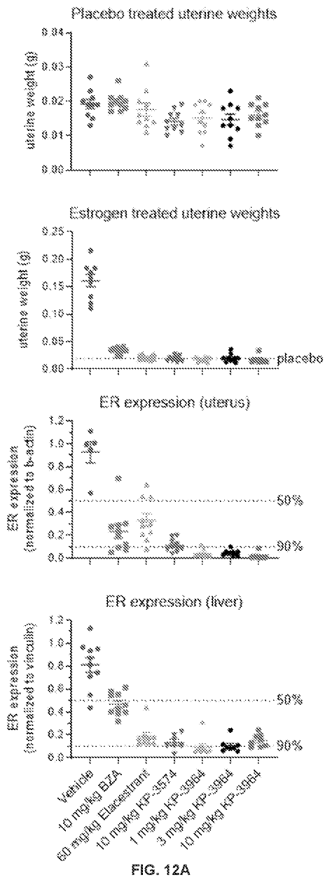

FIGS. 11A-11B: Dose impacts the effect of SERD fulvestrant on the growth of estrogen sensitive syngeneic A7C11 mouse mammary tumors. Left panel: 25 mg/kg fulvestrant dose not affect A7C11 tumor growth in mice lacking estrogen exposure, while escalation to 50 or 100 mg/kg fulvestrant resulted in tumor growth comparable to that observed for the estrogen control group. Middle panel: all doses (25, 50, or 100 mg/kg fulvestrant) similarly inhibited estrogen induction of A7C11 tumor growth, and also similarly repressed estrogen-dependent increases in uterine weight (right panel). Thus, exposure level presents a determinant of SERD activity, with higher exposure unexpectedly exacerbating, rather than mitigating, tumor growth. Thus, aspects other than ER turnover, including pharmacokinetics may affect SERD efficacy in vivo.

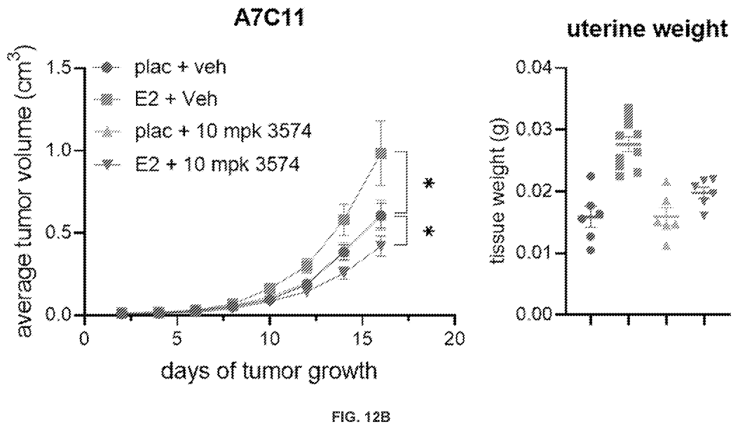

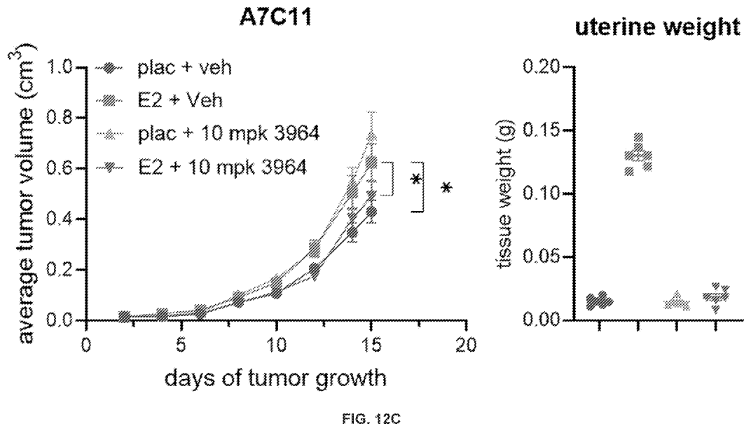

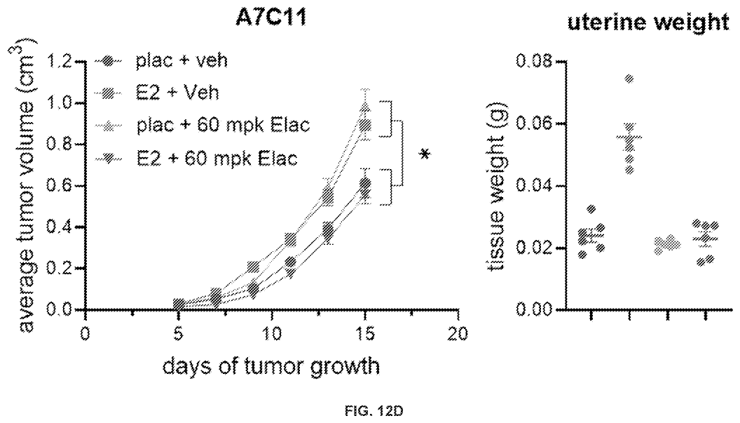

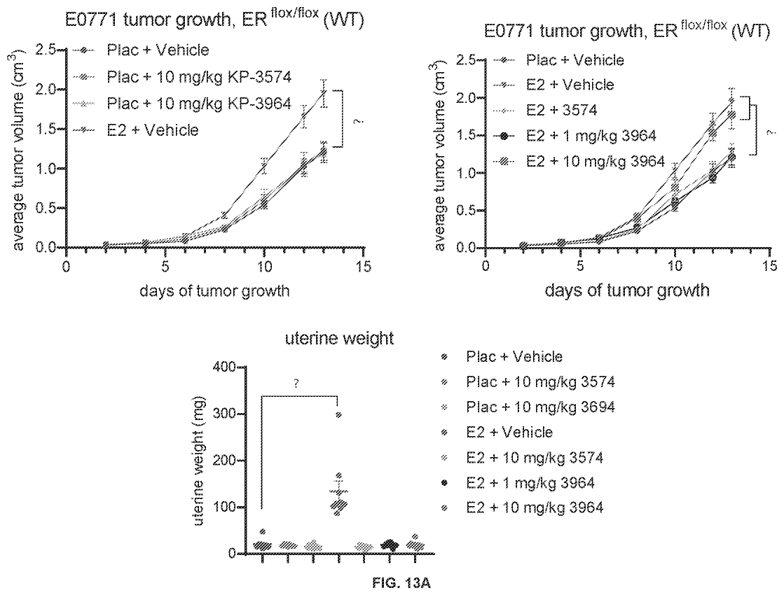

FIGS. 12A-12D: Brain excluded (KP-3574) and brain penetrant (KP-3964) SERDs exhibit activity and ER turnover in vivo similar to or exceeding benchmark SERDs bazedoxifene and elacestrant, using ER expression as a read out. However, KP-3574 did not impact tumor growth in animals lacking estrogen exposure, and in fact further repressed tumor growth when administered together with estrogen. Treatment with KP-3964 increased tumor growth in the absence of estrogen treatment, but paradoxically reversed estrogen stimulation of tumor growth, and this result was also observed for brain penetrant SERD elacestrant. Therefore, brain penetration may present a liability to SERD efficacy in the context of minimal estrogen exposure, as is common in menopause or following aromatase inhibition.

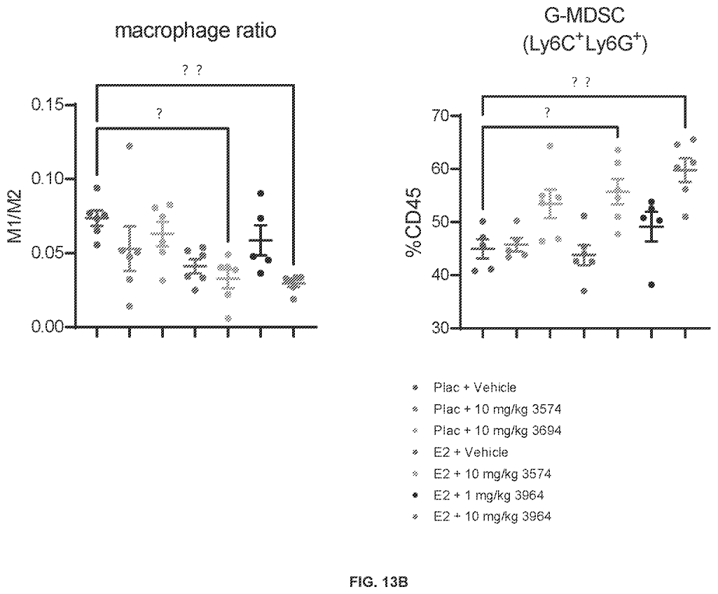

FIGS. 13A-13B: Evaluation of the brain penetrant SERD KP-3964 in a second syngeneic mouse mammary tumor model (E0771) shows that while the SERD itself does not increase tumor growth in the absence of estrogen treatment in this model, lower doses of the SERD are fully effective in reversing estrogen induced tumor growth, while the highest dose is ineffective, again indicating a non-linear relationship between dose and efficacy. Because brain exposure is proportional to dose administered, these data further support an unexpected tumor promoting role in the periphery for ER downregulation in the brain.

### Example 9

### Selective Estrogen Receptor Modulator (SERM) Lasofoxifene Co-Administered with Estradiol Represses A7C11 Mammary Tumor Growth

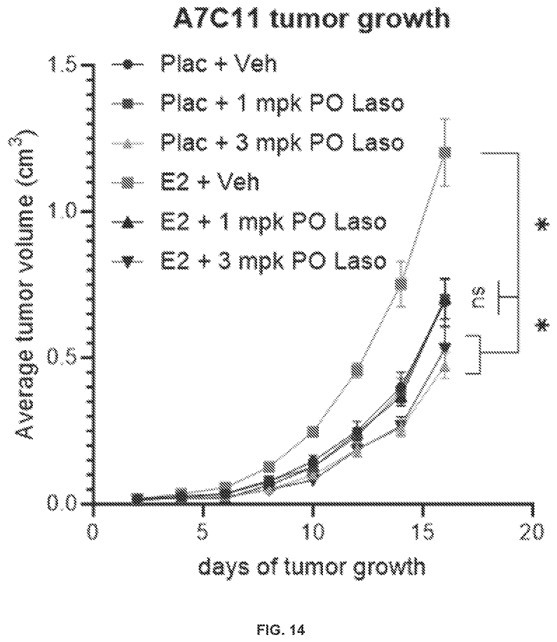

C57BL6/J mice were ovariectomized and assigned to treatment with a placebo or estradiol (2.72 mcg/mL) supplemented drinking water, begun 1 week prior to engrafting A7C11 mouse mammary tumors orthotopically. Two days after tumor initiation, the placebo and estrogen (E2) treatment groups were further subdivided to treatment co-administered with a vehicle or lasofoxifene (1 or 3 mg/kg po qd; orally administered daily). Presented in FIG. 14 are the average (+/−SEM) tumor volumes (cm³) recorded throughout tumor growth. A 2-way ANOVA followed by Tukey's multiple comparison test indicated an increase in A7C11 tumor growth when estradiol and the vehicle were co-administered. In contrast, a repression of tumor growth (as compared to the placebo/vehicle control group) was observed for mice receiving 3 mg/kg lasofoxifene co-administered with estradiol. Accordingly, the data in FIG. 14 show that lasofoxifene co-administered with estradiol represses A7C11 mammary tumor growth.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:
Clause 1. A method of treating cancer in a subject, the method comprising administering to the subject at least one brain-excluded estrogen receptor (ER) modulating drug such that the drug exposure in the brain is zero to minimal.
Clause 2. The method of clause 1, wherein the drug is administered at a dose and/or via a route such that the drug exposure in the brain is zero to minimal.
Clause 3. The method of any one of clauses 1-2, wherein the at least one brain-excluded ER modulating drug is administered to the subject at a dose that results in from zero to minimal brain exposure.
Clause 4. The method of clause 3, wherein the minimal brain exposure is 10:90 ratio drug concentration in CSF versus plasma.
Clause 5. The method of any one of clauses 1-4, wherein the dose results in no more than a 10% decrease in FES-PET signal intracranially relative to a control.
Clause 6. The method of any one of clauses 1-4, wherein the dose results in no more than a 20% decrease in FES-PET signal intracranially relative to a control.
Clause 7. The method of any one of clauses 1-6, wherein ER expression in the brain is not reduced relative to a control.
Clause 8. A method of inhibiting an estrogen receptor (ER) peripherally in a subject but not in the brain, the method comprising administering to the subject at least one brain-excluded estrogen receptor (ER) modulating drug such that the drug exposure in the brain is zero to minimal.
Clause 9. The method of clause 8, wherein the drug is administered at a dose and/or via a route such that the drug exposure in the brain is zero to minimal.
Clause 10. The method of any one of clauses 8-9, wherein the at least one brain-excluded ER modulating drug is administered to the subject at a dose that results in from zero to minimal brain exposure.
Clause 11. The method of clause 10, wherein the minimal brain exposure is 10:90 ratio drug concentration in CSF versus plasma.
Clause 12. The method of any one of clauses 8-11, wherein the dose results in no more than a 10% decrease in FES-PET signal intracranially relative to a control.
Clause 13. The method of any one of clauses 8-12, wherein the dose results in no more than a 20% decrease in FES-PET signal intracranially relative to a control.
Clause 14. The method of any one of clauses 8-13, wherein ER expression in the brain is not reduced relative to a control.
Clause 15. The method of any one of clauses 1-14, further comprising administering to the subject at least one brain-penetrating ER agonist or at least one brain-penetrating androgen receptor (AR) modulator.
Clause 16. The method of clause 15, wherein hot flashes in the subject are reduced.
Clause 17. The method of clause 16, wherein the duration, and/or intensity, and/or frequency of the hot flashes are reduced.
Clause 18. The method of any one of clauses 15-17, wherein the at least one brain-penetrating ER agonist comprises estrogen, estradiol, estetrol, estrone, conjugated estrogen, 10μ,17μ-dihydroxyestra-1,4-dien-3-one, 10μ,17α-Dihydroxyestra-1,4-dien-3-one, or a combination thereof.
Clause 19. The method of any one of clauses 15-18, wherein the at least one brain-excluded ER modulating drug and the at least one brain-penetrating ER agonist are administered simultaneously or sequentially.
Clause 20. The method of any one of clauses 15-17, wherein the at least one brain-penetrating AR modulator comprises testosterone, a selective AR modulator, DHEA, or androstendiol, or a combination thereof.
Clause 21. The method of any one of clauses 15-17 or 20, wherein the at least one brain-excluded ER modulating drug and the at least one brain-penetrating AR modulator are administered simultaneously or sequentially.
Clause 22. The method of any one of clauses 1-21, wherein the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof.
Clause 23. The method of clause 22, wherein the SERM is selected from lasofoxifene, bazodoxifene, tamoxifen, raloxifene, clomiphene, ospemiphene, arzoxifene, toremifene, and H3B6545, or a combination thereof.
Clause 24. The method of any one of clauses 22-23, wherein the SERD is selected from camizestrant, fulvestrant, LSZ102, LY3484356, giredestrant, GDC0927, D-052, AC0682, AZD9496, SAR439859, RAD1901, G1T48, Zn-c5, ARV-471, and OP-1250, or a combination thereof.
Clause 25. The method of any one of clauses 1-24, wherein the at least one brain-excluded ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months.
Clause 26. The method of any one of clauses 1-25, wherein the at least one brain-excluded ER modulating drug is administered to the subject for 1 year, 2 years, 3 years, 4 years, 5 years, or more than 5 years.
Clause 27. The method of any one of clauses 1-26, wherein the at least one brain-excluded ER modulating drug is administered to the subject orally, intravenously, transdermally, nasally, or vaginally.
Clause 28. The method of any one of clauses 1-27, wherein the ER is ER-alpha or ER-beta.
Clause 29. The method of any one of clauses 1-28, wherein the cancer is selected from breast cancer, melanoma, colon cancer, glioblastoma, ovarian cancer, head cancer, neck cancer, and lung cancer.
Clause 30. A composition for treating cancer, the composition comprising at least one brain-excluded estrogen receptor (ER) modulating drug and at least one brain-penetrating ER agonist.
Clause 31. A composition for treating cancer, the composition comprising at least one brain-excluded estrogen receptor (ER) modulating drug and at least one brain-penetrating androgen receptor (AR) modulator.
Clause 32. The composition of clause 30 or 31, wherein the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof.
Clause 33. The composition of clause 32, wherein the SERM is selected from lasofoxifene, bazodoxifene, tamoxifen, raloxifene, clomiphene, ospemiphene, arzoxifene, toremifene, and H3B6545, or a combination thereof.
Clause 34. The composition of any one of clauses 32-33, wherein the SERD is selected from fulvestrant, LSZ102, LY3484356, giredestrant, camizestrant, GDC0927, D-052,AC0682, AZD9496, SAR439859, RAD1901, G1T48, Zn-c5, ARV-471, and OP-1250, or a combination thereof.
Clause 35. The composition of any one of clauses 30 and 32-34, wherein the at least one brain-penetrating ER agonist comprises estrogen, estradiol, estetrol, estrone, conjugated estrogen, 10β,17β-dihydroxyestra-1,4-dien-3-one, 10β,17α-Dihydroxyestra-1,4-dien-3-one, or a combination thereof.
Clause 36. The composition of any one of clauses 31-35, wherein the at least one brain-penetrating AR modulator comprises testosterone, a selective AR modulator, DHEA, or androstendiol.
Clause 37. The composition of any one of clauses 30-36, wherein the cancer is selected from breast cancer, melanoma, colon cancer, glioblastoma, ovarian cancer, head cancer, neck cancer, and lung cancer.

1. A method of treating cancer in a subject, the method comprising administering to the subject at least one brain-excluded estrogen receptor (ER) modulating drug such that the drug exposure in the brain is zero to minimal.

2. The method of claim 1, wherein the drug is administered at a dose and/or via a route such that the drug exposure in the brain is zero to minimal.

3. The method of claim 1, wherein the at least one brain-excluded ER modulating drug is administered to the subject at a dose that results in from zero to minimal brain exposure.

4. The method of claim 3, wherein the minimal brain exposure is 10:90 ratio drug concentration in CSF versus plasma.

5. The method of claim 1, wherein the dose results in no more than a 10% decrease in FES-PET signal intracranially relative to a control.

6. The method of claim 1, wherein the dose results in no more than a 20% decrease in FES-PET signal intracranially relative to a control.

7. The method of claim 1, wherein ER expression in the brain is not reduced relative to a control.

8. A method of inhibiting an estrogen receptor (ER) peripherally in a subject but not in the brain, the method comprising administering to the subject at least one brain-excluded estrogen receptor (ER) modulating drug such that the drug exposure in the brain is zero to minimal.

9. The method of claim 8, wherein the drug is administered at a dose and/or via a route such that the drug exposure in the brain is zero to minimal.

10. The method of claim 8, wherein the at least one brain-excluded ER modulating drug is administered to the subject at a dose that results in from zero to minimal brain exposure.

11. The method of claim 10, wherein the minimal brain exposure is 10:90 ratio drug concentration in CSF versus plasma.

12. The method of claim 8, wherein the dose results in no more than a 10% decrease in FES-PET signal intracranially relative to a control.

13. The method of claim 8, wherein the dose results in no more than a 20% decrease in FES-PET signal intracranially relative to a control.

14. The method of claim 8, wherein ER expression in the brain is not reduced relative to a control.

15. The method of any one of claims 1-14, further comprising administering to the subject at least one brain-penetrating ER agonist or at least one brain-penetrating androgen receptor (AR) modulator.

16. The method of claim 15, wherein hot flashes in the subject are reduced.

17. The method of claim 16, wherein the duration, and/or intensity, and/or frequency of the hot flashes are reduced.

18. The method of any one of claims 15-17, wherein the at least one brain-penetrating ER agonist comprises estrogen, estradiol, estetrol, estrone, conjugated estrogen, 10β,17β-dihydroxyestra-1,4-dien-3-one, 10β,17α-Dihydroxyestra-1,4-dien-3-one, or a combination thereof.

19. The method of any one of claims 15-18, wherein the at least one brain-excluded ER modulating drug and the at least one brain-penetrating ER agonist are administered simultaneously or sequentially.

20. The method of any one of claims 15-17, wherein the at least one brain-penetrating AR modulator comprises testosterone, a selective AR modulator, DHEA, or androstendiol, or a combination thereof.

21. The method of any one of claims 15-17 or 20, wherein the at least one brain-excluded ER modulating drug and the at least one brain-penetrating AR modulator are administered simultaneously or sequentially.

22. The method of any one of claims 1-21, wherein the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof.

23. The method of claim 22, wherein the SERM is selected from lasofoxifene, bazodoxifene, tamoxifen, raloxifene, clomiphene, ospemiphene, arzoxifene, toremifene, and H3B6545, or a combination thereof.

24. The method of any one of claims 22-23, wherein the SERD is selected from camizestrant, fulvestrant, LSZ102, LY3484356, giredestrant, GDC0927, D-052, AC0682,AZD9496, SAR439859, RAD1901, G1T48, Zn-c5, ARV-471, and OP-1250, or a combination thereof.

25. The method of any one of claims 1-24, wherein the at least one brain-excluded ER modulating drug is administered to the subject once every day, once every 2 days, once every 3 days, once every 4 days, once every 5 days, once every 6 days, once every 7 days, once every week, once every 2 weeks, once every 3 weeks, once every 4 weeks, once every 5 weeks, once every 6 weeks, once every 7 weeks, once every 8 weeks, once every month, once every 2 months, once every 3 months, once every 4 months, once every 5 months, or once every 6 months.

26. The method of any one of claims 1-25, wherein the at least one brain-excluded ER modulating drug is administered to the subject for 1 year, 2 years, 3 years, 4 years, 5 years, or more than 5 years.

27. The method of any one of claims 1-26, wherein the at least one brain-excluded ER modulating drug is administered to the subject orally, intravenously, transdermally, nasally, or vaginally.

28. The method of any one of claims 1-27, wherein the ER is ER-alpha or ER-beta.

29. The method of any one of claims 1-28, wherein the cancer is selected from breast cancer, melanoma, colon cancer, glioblastoma, ovarian cancer, head cancer, neck cancer, and lung cancer, or a combination thereof.

30. A composition for treating cancer, the composition comprising at least one brain-excluded estrogen receptor (ER) modulating drug and at least one brain-penetrating ER agonist.

31. A composition for treating cancer, the composition comprising at least one brain-excluded estrogen receptor (ER) modulating drug and at least one brain-penetrating androgen receptor (AR) modulator.

32. The composition of claim 30 or 31, wherein the at least one brain-excluded ER modulating drug is selected from a selective estrogen receptor modulator (SERM), a selective estrogen receptor degrader/downregulator (SERD), CERAN, aromatase inhibitor, GNRH agonist, or a combination thereof.

33. The composition of claim 32, wherein the SERM is selected from lasofoxifene, bazodoxifene, tamoxifen, raloxifene, clomiphene, ospemiphene, arzoxifene, toremifene, and H3B6545, or a combination thereof.

34. The composition of any one of claims 32-33, wherein the SERD is selected from fulvestrant, LSZ102, LY3484356, giredestrant, camizestrant, GDC0927, D-052, AC0682,AZD9496, SAR439859, RAD1901, G1T48, Zn-c5, ARV-471, and OP-1250, or a combination thereof.

35. The composition of any one of claims 30 and 32-34, wherein the at least one brain-penetrating ER agonist comprises estrogen, estradiol, estetrol, estrone, conjugated estrogen, 10β,17β-dihydroxyestra-1,4-dien-3-one, 10β,17α-Dihydroxyestra-1,4-dien-3-one, or a combination thereof.

36. The composition of any one of claims 31-35, wherein the at least one brain-penetrating AR modulator comprises testosterone, a selective AR modulator, DHEA, or androstendiol.

37. The composition of any one of claims 30-36, wherein the cancer is selected from breast cancer, melanoma, colon cancer, glioblastoma, ovarian cancer, head cancer, neck cancer, and lung cancer, or a combination thereof.
